SEQUENCE LISTING (47 sequences)

SEQ ID NO: 1 - protein, 19 aa, Artificial Sequence
  signal peptide
MRFPSIFTAVLFAASSALA

SEQ ID NO: 2 - DNA, 57 bp, Artificial Sequence
  signal peptide
atgagattcccatccatcttcactgctgttttgttcgctgcttcttctgctttggct

SEQ ID NO: 3 - DNA, 1491 bp, Artificial Sequence
  synthetic gene
ttgccaatgattgacaagcacttgccatcctctattggtcaatcctcctacaagacttccagagaaagagctgaggctgttaaggacgcttttagattcgcttggaacggttacttggaacacgctttcccaaacgatgaattgcacccagtttccaacactccaggaaactctagaaacggttggggagcttctgctgttgatgctttgtccactgctatcatcatggacatgccagacgttgttgagaagattttggaccacatctccaacattgactactcccagactgacactatgtgttccttgttcgagactactatcagatacttgggtggaatgatctctgcttacgacttgttgaagggtccaggttctcacttggtttctgacccagctaaggttgacgttttgttggctcagtccttgaagttggctgacgttttgaagttcgctttcgacactaagactggtattccagctaacgagttgaacatcactgacaagtctactgacggttccactacaaacggattggctactactggtactttggttttggagtggactagattgtccgacattactggtgatccagagtacggtagattggctcaaaagggtgagtcctacttgttgaacccacaaccatcttcctctgaaccattcccaggattggttggtagaactatcgacatcgagactggtttgttcagagatgactacgtttcttggggtggtggttctgactctttctacgagtacttgatcaagatgtacgtttacgacaaggacagattcggtaagtacaaagagagatgggttacagctgctgagtccactatcaagcacttgaagtcctctccatccactagaaaggacttgactttcgttgctacttactccggtggtagattgggattgaactccggtcacttgacttgtttcgacggtggtaacttcttgttgggtggtcaaatcttggacagagatgacttcactaagttcggattggagttggttgaaggatgttacgctacttacgctgctactgctactaagattggtccagagggttttggatgggacgatactaaggttccagaagctcaggctgagttctacaaagaggctggtttctacatcactacttcctactacaacttgagaccagaggttatcgagtccatctactacgcttacagaatgactaaggacccaaagtaccaagaatgggcttgggatgcttttgttgctatcaacgctactactagaacttccactggtttcactgctatcggtgacgttaacactccagacggtggtagaaagtacgacaaccaagagtccttcttgttcgctgaggttatgaagtactcctacttgattcactctccagaagctgattggcaagttgctggtccaggtggtactaatgcttacgttttcaacactgaggctcacccagttaaggttttctccagaggttgt

SEQ ID NO: 4 - protein, 497 aa, Coccidioides immitis
LPMIDKHLPSSIGQSSYKTSRERAEAVKDAFRFAWNGYLEHAFPNDELHPVSNTPGNSRNGWGASAVDALSTAIIMDMPDVVEKILDHISNIDYSQTDTMCSLFETTIRYLGGMISAYDLLKGPGSHLVSDPAKVDVLLAQSLKLADVLKFAFDTKTGIPANELNITDKSTDGSTTNGLATTGTLVLEWTRLSDITGDPEYGRLAQKGESYLLNPQPSSSEPFPGLVGRTIDIETGLFRDDYVSWGGGSDSFYEYLIKMYVYDKDRFGKYKERWVTAAESTIKHLKSSPSTRKDLTFVATYSGGRLGLNSGHLTCFDGGNFLLGGQILDRDDFTKFGLELVEGCYATYAATATKIGPEGFGWDDTKVPEAQAEFYKEAGFYITTSYYNLRPEVIESIYYAYRMTKDPKYQEWAWDAFVAINATTRTSTGFTAIGDVNTPDGGRKYDNQESFLFAEVMKYSYLIHSPEADWQVAGPGGTNAYVFNTEAHPVKVFSRGC

SEQ ID NO: 5 - DNA, 1491 bp, Artificial Sequence; identical to SEQ ID NO: 3
  synthetic gene

SEQ ID NO: 6 - protein, 497 aa, Coccidioides posadasii
LPMIDKDLPSSISQSSDKTSQERAEAVKDAFRFAWEGYLEHAFPNDELHPVSNTPGNSRNGWGASAVDALSTAIIMDMPDVVEKILDHISNIDYSQTDTMCSLFETTIRYLGGMISAYDLLKGPGSHLVSDPAKVDVLLAQSLKLADVLKFAFDTKTGIPANELNITDKSTDGSTTNGLATTGTLVLEWTRLSDITGDPEYGRLAQKGESYLLNPQPSSSEPFPGLVGRTIDIETGLFRDDYVSWGGGSDSFYEYLIKMYVYDKGRFGKYKDRWVTAAESTIEHLKSSPSTRKDLTFVATYSGGRLGLNSGHLTCFDGGNFLLGGQILNRDDFTKFGLELVEGCYATYAATATKIGPEGFGWDATKVPEAQAEFYKEAGFYITTSYYNLRPEVIESIYYAYRMTKDPKYQEWAWDAFVAINATTRTSTGFTAIGDVNTPDGGRKYDNQESFLFAEVMKYSYLIHSPEADWQVAGPGGTNAYVFNTEAHPVKVFSRGC

SEQ ID NO: 7 - DNA, 1473 bp, Artificial Sequence
  synthetic gene
cacccatacggtgaaactgaggctgttttgagatccgaaccaaagtccaaccaagctaaggctgacgctgttaaagaggcttttcagcacgcttggaacggttacatgaagtacgctttcccacacgatgaattgactccagtttccaacggtcacgctgattctagaaatggatggggagcttctgctgttgatgctttgtccactgctgttatcatgggaaaggctgatgttgttaacgctatcttggagcacgttgctgacattgacttctccaagacttccgacactgtttccttgttcgagactactatcagatacttggctggaatgttgtctggttacgacttgttgcaaggtccagctaagaacttggttgacaaccaggacttgatcgatggtttgttggaccagtccagaaacttggctgacgttttgaagttcgctttcgacactccatctggtgttccatacaacaacatcaacatcacttcccacggtaatgatggtgctactacaaacggattggctgttactggtactttggttttggagtggactagattgtctgacttgactggtgacgaagagtacgctaagttgtcccaaaaggctgagtcctacttgttgaagccacaaccatcttcctctgaaccattcccaggattggttggttcctccatcaacattaacgacggtcaattcgctgactctagagtttcttggaacggtggtgacgactctttctacgagtacttgatcaagatgtacgtttacgacccaaagagattcgagacttacaaggacagatgggttttggctgctgagtccactattaagcacttgaagtcccacccaaagtctagaccagacttgactttcttgtcctcctactccaacagaaactacgacttgtcctcccaacacttgacttgtttcgacggtggttcctttttgttgggtggtactgttttggacagacaggacttcattgacttcggattggagttggttgatggttgtgaggctacttacaactccactttgactaagattggtccagattcttggggatgggacccaaagaaggttccatccgaccagaaagagttctacgagaaggctggtttctacatttcttccggttcctacgttttgagaccagaggttatcgagtccttctactacgctcacagagttactggtaaagaaatctacagagactgggtttggaacgctttcgttgctatcaactccacttgtagaactgactctggtttcgctgctgtttccgacgttaacaaggctaacggtggttctaagtacgacaaccaagagtccttcttgttcgctgaggttatgaagtactcctacttggctcactctgaagatgctgcttggcaagttcaaaagggtggaaagaacactttcgtttacaacactgaggctcacccaatttccgttgctagaaac

SEQ ID NO: 8 - protein, 491 aa, Penicillium citrinum
HPYGETEAVLRSEPKSNQAKADAVKEAFQHAWNGYMKYAFPHDELTPVSNGHADSRNGWGASAVDALSTAVIMGKADVVNAILEHVADIDFSKTSDTVSLFETTIRYLAGMLSGYDLLQGPAKNLVDNQDLIDGLLDQSRNLADVLKFAFDTPSGVPYNNINITSHGNDGATTNGLAVTGTLVLEWTRLSDLTGDEEYAKLSQKAESYLLKPQPSSSEPFPGLVGSSININDGQFADSRVSWNGGDDSFYEYLIKMYVYDPKRFETYKDRWVLAAESTIKHLKSHPKSRPDLTFLSSYSNRNYDLSSQHLTCFDGGSFLLGGTVLDRQDFIDFGLELVDGCEATYNSTLTKIGPDSWGWDPKKVPSDQKEFYEKAGFYISSGSYVLRPEVIESFYYAHRVTGKEIYRDWVWNAFVAINSTCRTDSGFAAVSDVNKANGGSKYDNQESFLFAEVMKYSYLAHSEDAAWQVQKGGKNTFVYNTEAHPISVARN

SEQ ID NO: 9 - DNA, 1518 bp, Artificial Sequence
  synthetic gene
tccccattgactaagagagctatccagtactctccaaacccagaaaaggctgaggctgttaaggctgcttttagaagatcctgggacggttacatgcaacatgctttcccacacgacactttgagaccagtttctcaaggatgggctgatgatagaaatggatggggagcttctgctgttgatgctttcggtactgctatcgttatggacgaagaggctatcgttcagcaaatcttggacttcgttccaactatcgacttcgacaagacttctactgaggtttccttgttcgagactactatcagatacttgggaggattggtttctgcttacgacttgttgactgagccaaacggtaaacacgctactggtaacgagactttggttaaggctgttttgtcccaggctgagagattggctacaaacttgaagttcgctttcgacactccatctggtattccagacaactccgtttacttcaacccatccagaagaggaggttcccaaactaacggtatcgctactatcggtactttgcagttggagtggactagattgggagacttgactggtaacccagttttcactgagttggctttgaaggctaactcctacttgttggacccaaagccagctttgggtgaaccattccctggtttgttgggaactaacgttaacatcactactggtttgttcactgacggtaatggtggatggggtggtggtactgactctttctacgagtacttgatcaagatgtacttgtacgacccagagagattcggtcaatacagagacagatgggttttggctgctgactcctccattaagtacatggcttcccacccatctactagaccagacttgactttcttggctgagtggagagacacttccttgagatacatctcccagcacttggcttgttttgacggtggtaacttcatcttgggaggattgactttgcagaaccagaagtacgttgacttcggattggctttgactaacgcttgtcacgagacttacatccagactgttactggtattggtccagaggttttcagatggcaagatgcttctgctgctaactcttccactaatccacctgctccagctggattgcaagagttctacaagagagctggtttctggattccaccatccaacgatattggtggtgctgctatctaccaattgagaccagaggttatcgagtccttctactacgcttacagagctactggtgacactaagtaccaagactgggcttgggaggctttcttgtccatcaacagaacttgtgctgctggtactgcttactcttccatctccgacgttaatgctccaggtggtggttcttctttggacttccaagagtccttttggttcgctgagactatgaagtactcctacttgattcacgctgaagatgctccatggcaagttaaggctgacagaactaacggatgggttttcaacactgaggctcacccaattaaggttcacggtagaccaggt

SEQ ID NO: 10 - protein, 506 aa, Magnaporthe grisea
SPLTKRAIQYSPNPEKAEAVKAAFRRSWDGYMQHAFPHDTLRPVSQGWADDRNGWGASAVDAFGTAIVMDEEAIVQQILDFVPTIDFDKTSTEVSLFETTIRYLGGLVSAYDLLTEPNGKHATGNETLVKAVLSQAERLATNLKFAFDTPSGIPDNSVYFNPSRRGGSQTNGIATIGTLQLEWTRLGDLTGNPVFTELALKANSYLLDPKPALGEPFPGLLGTNVNITTGLFTDGNGGWGGGTDSFYEYLIKMYLYDPERFGQYRDRWVLAADSSIKYMASHPSTRPDLTFLAEWRDTSLRYISQHLACFDGGNFILGGLTLQNQKYVDFGLALTNACHETYIQTVTGIGPEVFRWQDASAANSSTNPPAPAGLQEFYKRAGFWIPPSNDIGGAAIYQLRPEVIESFYYAYRATGDTKYQDWAWEAFLSINRTCAAGTAYSSISDVNAPGGGSSLDFQESFWFAETMKYSYLIHAEDAPWQVKADRTNGWVFNTEAHPIKVHGRPG

SEQ ID NO: 11 - DNA, 1476 bp, Artificial Sequence
  synthetic gene
tacccacatttcggttcctctcagccagttttgcactcttcttccgacactactcaatctagagctgacgctatcaaggctgctttttctcatgcttgggacggttacttgcaatacgctttcccacacgatgaattgcacccagtttccaacggttacggtgactctagaaatggatggggagcttctgctgttgatgctttgtccactgctgttatcatgagaaacgctactatcgttaaccaaatcttggaccatgttggaaagatcgactactccaagactaacactactgtttccttgttcgagactactatcagatacttgggtggaatgttgtctggttacgacttgttgaagggtccagtttccgatttggttcagaactcctccaagatcgacgttttgttgactcagtccaagaacttggctgacgttttgaagttcgctttcgacactccatctggtgttccatacaacaacttgaacatcacttccggtggtaatgatggtgctaagactaacggattggctgttactggtactttggctttggagtggactagattgtctgacttgactggtgacactacttacgctgacttgtcccaaaaggctgagtcctacttgttgaacccacaaccaaagtctgctgaaccattcccaggattggttggttccaacatcaacatctccaacggtcaattcactgacgctcaggtttcatggaatggtggtgacgactcttactacgagtacttgatcaagatgtacgtttacgacccaaagagattcggtttgtacaaggacagatgggttgctgctgctcaatctactatgcagcacttggcttctcatccatcttctagaccagacttgactttcttggcttcctacaacaacggtactttgggtttgtcctcccagcatttgacttgtttcgacggtggttcctttttgttgggtggtactgttttgaacagaactgacttcatcaacttcggattggacttggtttctggttgtcacgacacttacaactccactttgactggtattggtccagaatctttctcttgggacacttccgacattccatcttcccaacagtccttgtacgaaaaggctggtttctacatcacttctggtgcttacatcttgagaccagaggttatcgagtccttctactacgcttggagagttactggtcaagagacttacagagactggatttggtctgctttctccgctgttaacgactactgtagaacttcttccggtttctccggattgactgacgttaatgctgctaatggtggttccagatacgacaaccaagagtccttcttgttcgctgaggttatgaagtactcctacatggctttcgctgaagatgctgcttggcaagttcaaccaggttccggtaaccagttcgttttcaacactgaggctcacccagttagagtttcttccact

SEQ ID NO: 12 - protein, 492 aa, Aspergillus saitoi
YPHFGSSQPVLHSSSDTTQSRADAIKAAFSHAWDGYLQYAFPHDELHPVSNGYGDSRNGWGASAVDALSTAVIMRNATIVNQILDHVGKIDYSKTNTTVSLFETTIRYLGGMLSGYDLLKGPVSDLVQNSSKIDVLLTQSKNLADVLKFAFDTPSGVPYNNLNITSGGNDGAKTNGLAVTGTLALEWTRLSDLTGDTTYADLSQKAESYLLNPQPKSAEPFPGLVGSNINISNGQFTDAQVSWNGGDDSYYEYLIKMYVYDPKRFGLYKDRWVAAAQSTMQHLASHPSSRPDLTFLASYNNGTLGLSSQHLTCFDGGSFLLGGTVLNRTDFINFGLDLVSGCHDTYNSTLTGIGPESFSWDTSDIPSSQQSLYEKAGFYITSGAYILRPEVIESFYYAWRVTGQETYRDWIWSAFSAVNDYCRTSSGFSGLTDVNAANGGSRYDNQESFLFAEVMKYSYMAFAEDAAWQVQPGSGNQFVFNTEAHPVRVSST

SEQ ID NO: 13 - DNA, 1467 bp, Artificial Sequence
  synthetic gene
tacccacaattcaagttcgagcagagagttgctagatccaactcttctgagtccagagctaacgctgttaaagaggcttttgttcacgcttgggacggttacatgcaatacgcttacccacacgatgaattgcacccaatctccaacggtgttggtgattctagaaacggatggggagcttctgctgttgatgctttgtccactgctgttatcatgggtaacgagactatcgttaaccaaatcttggaccacatcgctactattgactactccaagactgacgaccaagtttccttgttcgagactactatcagatacttgggtggaatgttgtctggttacgacttgttgaagggtccagcttccaacttggttaaggaccaggctaaggttaagactttgttggaccagtcccaaaacttggctgacgttttgaagttcgctttcgacactccatctggtatcccatacaacaacatcaacatcacttcccacggtaatgatggtgctactacaaacggattggctgttactggtactttggttttggagtggactagattgtctgacttgactggtgacactgagtacgctcaattgtcccaaaaggctgaggactacttgttgaacccatctccaaagtctgctgaaccattcgaaggattggttggttcccacatcaacatttccaacggtgctttcgctgatggtcaagtttcttggaacggtggtgacgactctttctacgagtacttgatcaagatgtacgtttacgacccaaagagattctctacttacggtgacagatgggttaaggctgctgagtcctccattaagcacttggcttctcacccagaaaagagaccagacttgactttcttggcttcctacaacgatggtcagtacggtttgtcctcccaacacttgacttgtttcgacggtggttcctttttgttgggtggtactgttttggacagagatgacttcatccagttcggattggatttggttaagggttgtcacgagacttacaaccagactttgactggtattggtccagaatctttcggatgggacccaaagaatgttccatccgaccagaaagagttgtacgagagagctggtttctacatttcctccggtgcttacattttgagaccagaggttatcgagtccttctactacgcttggagaatcactggacaagaaatctacagagaatgggtttggaacgctttcgttaacatcaacaagtactgtagaactgactctggtttcgctggattgactaacgttaacgctgctaacggtggtggaagatacgacaaccaagagtccttcttgttcgctgaggttttgaaatacgtttacttgactttcgctccagacaacgagtggcaagttcaaagaggaaagggtaacaagttcgtttacaacactgaggctcacccagttagagttgctgct

SEQ ID NO: 14 - protein, 489 aa, Aspergillus oryzae
YPQFKFEQRVARSNSSESRANAVKEAFVHAWDGYMQYAYPHDELHPISNGVGDSRNGWGASAVDALSTAVIMGNETIVNQILDHIATIDYSKTDDQVSLFETTIRYLGGMLSGYDLLKGPASNLVKDQAKVKTLLDQSQNLADVLKFAFDTPSGIPYNNINITSHGNDGATTNGLAVTGTLVLEWTRLSDLTGDTEYAQLSQKAEDYLLNPSPKSAEPFEGLVGSHINISNGAFADGQVSWNGGDDSFYEYLIKMYVYDPKRFSTYGDRWVKAAESSIKHLASHPEKRPDLTFLASYNDGQYGLSSQHLTCFDGGSFLLGGTVLDRDDFIQFGLDLVKGCHETYNQTLTGIGPESFGWDPKNVPSDQKELYERAGFYISSGAYILRPEVIESFYYAWRITGQEIYREWVWNAFVNINKYCRTDSGFAGLTNVNAANGGGRYDNQESFLFAEVLKYVYLTFAPDNEWQVQRGKGNKFVYNTEAHPVRVAA

SEQ ID NO: 15 - DNA, 1689 bp, Artificial Sequence
  synthetic gene
ttgccaagattggaagatggtggtagaccaggtgctggtagagaagattctttctggtctaaggttccattgcactacccaccagactctattagaccattgccaactggattgccagttcaatacccaccaattcaaactactactttcccagctgaagaagatcaagctgctagaggtagaagagttcaaagacaaaaggctgttagagatgttttcgctaagtgttgggcttcttacagaaagttggcttggggtgctgatgaattgactccagtttctggtggtagaaaggacccatttggtggatggtcagctactttggttgactctttggacactttgtggattatggacatgaaaactgaatttgacgaagctgttgctgctgctgacggtattaacttcactcacactgctgttgacagagttaacgttttcgaaactaacattagataccttggtggtttcttggctgctttcgacttgtctggtgacaagagattgttgggaaaggctacagaggtcggagacatgttgtacaaggctttcgacactccatctcacatgccaattactagatgggacatgagagctgctattaagggtgctaaacaagttgcttctgaaggattgattgctgaagtcggaactttgtctatggagttcactagattgtctatgttgactggtgacccaaagtggttcgacttggctcaaagaattactgacgacatggctgctcaacaagactctactgctgttcctggtttgtggccattgaaggttaacggtcaaaagagaattttcaactctggtccagactttactttgggtgctatggctgactctgcttacgaatacttgccaaagatgtctgctatgatgggtggtcaattgccaatgtaccaaactatgtacgaaaaggctatggacgctgctactaagcacaacttgttcagaccaatgactccaactaacgaagatattttgattgctggtgacgttcacgctagagaaaacggaatggaatttgaagctagaggtcaacacttgacttgtttccttggtggattgatggctttgggtggtagattgtacggtagagaaaaggacgttattgctggtagaaagttgactgacggttgtgtttggacttataaagcatttccacaaggtattatgccagaatctttcgttactgtttcttgtccagctggtgatgcttgtgaatgggacgagggcgtctggaagaaagaagttttgagaaaggctaacaaggatactgacggtccagaatctaacgctgaagctgaagttatcattaagaagcaaagattgccacaaggtttcacagctgtgttcgacagagcttacttgttgagaccagaagctattgaatctgttttcgttttgtacagagttactggaaaggttgaattgttggaagctgcttgggatatgttcactgctattgacaaggctacatctacagacttggctaattctgctatcagggacgtcacatccactgataagccagaggctaaagactctatggaatctttctggatggctgaaactttgaagtacttctacttgattttctctgacccagaattgattaacttgaacgaatacgttttgaacactgaagctcacccattcagaagattgttgccatctcca

SEQ ID NO: 16 - protein, 563 aa, Chaetomium globosum
LPRLEDGGRPGAGREDSFWSKVPLHYPPDSIRPLPTGLPVQYPPIQTTTFPAEEDQAARGRRVQRQKAVRDVFAKCWASYRKLAWGADELTPVSGGRKDPFGGWSATLVDSLDTLWIMDMKTEFDEAVAAADGINFTHTAVDRVNVFETNIRYLGGFLAAFDLSGDKRLLGKATEVGDMLYKAFDTPSHMPITRWDMRAAIKGAKQVASEGLIAEVGTLSMEFTRLSMLTGDPKWFDLAQRITDDMAAQQDSTAVPGLWPLKVNGQKRIFNSGPDFTLGAMADSAYEYLPKMSAMMGGQLPMYQTMYEKAMDAATKHNLFRPMTPTNEDILIAGDVHARENGMEFEARGQHLTCFLGGLMALGGRLYGREKDVIAGRKLTDGCVWTYKAFPQGIMPESFVTVSCPAGDACEWDEGVWKKEVLRKANKDTDGPESNAEAEVIIKKQRLPQGFTAVFDRAYLLRPEAIESVFVLYRVTGKVELLEAAWDMFTAIDKATSTDLANSAIRDVTSTDKPEAKDSMESFWMAETLKYFYLIFSDPELINLNEYVLNTEAHPFRRLLPSP

SEQ ID NO: 17 - DNA, 49 bp, Artificial Sequence
  primer
gagctcggccttggaggccgcggaaacggcagtaaacaatggagcttca

SEQ ID NO: 18 - DNA, 38 bp, Artificial Sequence
  primer
ggccggccttatgtagcgtttctgataaattgtgtgat

SEQ ID NO: 19 - DNA, 699 bp, Artificial Sequence
  partial vector
gagctcggccttggaggccgcggaaacggcagtaaacaatggagcttcattagtgggtgttattatggtccctggccgggaacgaacggtgaaacaagaggttgcgagggaaatttcgcagatggtgcgggaaaagagaatttcaaagggctcaaaatacttggattccagacaactgaggaaagagtgggacgactgtcctctggaagactggtttgagtacaacgtgaaagaaataaacagcagtggtccatttttagttggagtttttcgtaatcaaagtatagatgaaatccagcaagctatccacactcatggtttggatttcgtccaactacatgggtctgaggattttgattcgtatatacgcaatatcccagttcctgtgattaccagatacacagataatgccgtcgatggtcttaccggagaagacctcgctataaatagggccctggtgctactggacagcgagcaaggaggtgaaggaaaaaccatcgattgggctcgtgcacaaaaatttggagaacgtagaggaaaatatttactagccggaggtttgacacctgataatgttgctcatgctcgatctcatactggctgtattggtgttgacgtctctggtggggtagaaacaaatgcctcaaaagatatggacaagatcacacaatttatcagaaacgctacataaggccggcc

SEQ ID NO: 20 - DNA, 34 bp, Artificial Sequence
  primer
actagtatttaaataagtcaattaaatacacgct

SEQ ID NO: 21 - DNA, 48 bp, Artificial Sequence
  primer
gtcgacggccaagacggccaagtcgtatctcctttccacgacatccca

SEQ ID NO: 22 - DNA, 867 bp, Artificial Sequence
  partial vector
actagtatttaaataagtcaattaaatacacgcttgaaaggacattacatagctttcgatttaagcagaaccagaaatgtagaaccacttgtcaatagattggtcaatcttagcaggagcggctgggctagcagttggaacagcagaggttgctgaaggtgagaaggatggagtggattgcaaagtggtgttggttaagtcaatctcaccagggctggttttgccaaaaatcaacttctcccaggcttcacggcattcttgaatgacctcttctgcatacttcttgttcttgcattcaccagagaaagcaaactggttctcaggttttccatcagggatcttgtaaattctgaaccattcgttggtagctctcaacaagcccggcatgtgcttttcaacatcctcgatgtcattgagcttaggagccaatgggtcgttgatgtcgatgacgatgaccttccagtcagtctctccctcatccaacaaagccataacaccgaggaccttgacttgcttgacctgtccagtgtaacctacggcttcaccaatttcgcaaacgtccaatggatcattgtcacccttggccttggtctctggatgagtgacgttagggtcttcccatgtctgagggaaggcaccgtagttgtgaatgtatccgtggtgagggaaacagttacgaacgaaacgaagttttcccttctttgtgtcctgaagaattgggttcagtttctcctccttggaaatctccaacttggcgttggtccaacgggggacttcaacaaccatgttgagaaccttcttggattcgtcagcataaagtgggatgtcgtggaaaggagatacgacttggccgtcttggccgtcgac

SEQ ID NO: 23 - DNA, 32 bp, Artificial Sequence
  primer
ggccggccatttacaattagtaatattaaggt

SEQ ID NO: 24 - DNA, 30 bp, Artificial Sequence
  primer
gtttaaacctactaagcgacgaaaacggga

SEQ ID NO: 25 - DNA, 392 bp, Artificial Sequence
  transcriptional terminator
ggccggccatttacaattagtaatattaaggtggtaaaaacattcgtagaattgaaatgaattaatatagtatgacaatggttcatgtctataaatctccggcttcggtaccttctccccaattgaatacattgtcaaaatgaatggttgaactattaggttcgccagtttcgttattaagaaaactgttaaaatcaaattccatatcatcggttccagtgggaggaccagttccatcgccaaaatcctgtaagaatccattgtcagaacctgtaaagtcagtttgagatgaaatttttccggtctttgttgacttggaagcttcgttaaggttaggtgaaacagtttgatcaaccagcggctcccgttttcgtcgcttagtaggtttaaac

SEQ ID NO: 26 - DNA, 954 bp, Artificial Sequence
  promoter
ctcgagagatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacggcggccgc

SEQ ID NO: 27 - DNA, 300 bp, Artificial Sequence
  terminator
ttaattaaacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgcaagctgccggctcttaa

SEQ ID NO: 28 - DNA, 1353 bp, Artificial Sequence
  Antibody heavy chain
gaggttcagttggttgaatctggaggaggattggttcaacctggtggttctttgagattgtcctgtgctgcttccggtttcaacatcaaggacacttacatccactgggttagacaagctccaggaaagggattggagtgggttgctagaatctacccaactaacggttacacaagatacgctgactccgttaagggaagattcactatctctgctgacacttccaagaacactgcttacttgcagatgaactccttgagagctgaggatactgctgtttactactgttccagatggggtggtgatggtttctacgctatggactactggggtcaaggaactttggttactgtttcctccgcttctactaagggaccatctgttttcccattggctccatcttctaagtctacttccggtggtactgctgctttgggatgtttggttaaagactacttcccagagccagttactgtttcttggaactccggtgctttgacttctggtgttcacactttcccagctgttttgcaatcttccggtttgtactctttgtcctccgttgttactgttccatcctcttccttgggtactcagacttacatctgtaacgttaaccacaagccatccaacactaaggttgacaagaaggttgagccaaagtcctgtgacaagactcatacttgtccaccatgtccagctccagaattgttgggtggtccttccgtttttttgttcccaccaaagccaaaggacactttgatgatctccagaactccagaggttacatgtgttgttgttgacgtttctcacgaggacccagaggttaagttcaactggtacgttgacggtgttgaagttcacaacgctaagactaagccaagagaggagcagtacaactccacttacagagttgtttccgttttgactgttttgcaccaggattggttgaacggaaaggagtacaagtgtaaggtttccaacaaggctttgccagctccaatcgaaaagactatctccaaggctaagggtcaaccaagagagccacaggtttacactttgccaccatccagagatgagttgactaagaaccaggtttccttgacttgtttggttaagggattctacccatccgacattgctgttgaatgggagtctaacggtcaaccagagaacaactacaagactactccacctgttttggactctgacggttcctttttcttgtactccaagttgactgttgacaagtccagatggcaacagggtaacgttttctcctgttccgttatgcatgaggctttgcacaaccactacactcaaaagtccttgtctttgtcccctggtaagtaa

SEQ ID NO: 29 - protein, 450 aa, Artificial Sequence
  Antibody heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - DNA, 645 bp, Artificial Sequence
  Antibody light chain
gacatccaaatgactcaatccccatcttctttgtctgcttccgttggtgacagagttactatcacttgtagagcttcccaggacgttaatactgctgttgcttggtatcaacagaagccaggaaaggctccaaagttgttgatctactccgcttccttcttgtactctggtgttccatccagattctctggttccagatccggtactgacttcactttgactatctcctccttgcaaccagaagatttcgctacttactactgtcagcagcactacactactccaccaactttcggacagggtactaaggttgagatcaagagaactgttgctgctccatccgttttcattttcccaccatccgacgaacagttgaagtctggtacagcttccgttgtttgtttgttgaacaacttctacccaagagaggctaaggttcagtggaaggttgacaacgctttgcaatccggtaactcccaagaatccgttactgagcaagactctaaggactccacttactccttgtcctccactttgactttgtccaaggctgattacgagaagcacaaggtttacgcttgtgaggttacacatcagggtttgtcctccccagttactaagtccttcaacagaggagagtgttaa

SEQ ID NO: 31 - protein, 213 aa, Artificial Sequence
  Antibody light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLNNFYPREAKVQWKVDNALQSGNSQESVTEQDSKDSTYSLSSTLTLSKADYEKHKVYACEVTHQGLSSPVTKSFNRGEC

SEQ ID NO: 32 - DNA, 25 bp, Artificial Sequence
  primer
ggaagctcgagtttaagcggtgctg

SEQ ID NO: 33 - DNA, 27 bp, Artificial Sequence
  primer
gattgcctcctgattacgaagaagccg

SEQ ID NO: 34 - DNA, 26 bp, Artificial Sequence
  primer
ctactaagcgacgaaaacgggagccg

SEQ ID NO: 35 - DNA, 27 bp, Artificial Sequence
  primer
gggagagttgaaggttgtattattgcc

SEQ ID NO: 36 - DNA, 702 bp, Artificial Sequence
  Antibody light chain
atgagattcccatccatcttcactgctgttttgttcgctgcttcttctgctttggctgacattcagatgacacagtccccatctactttgtctgcttccgttggtgacagagttactatcacttgtaagtgtcagttgtccgttggttacatgcactggtatcagcaaaagccaggaaaggctccaaagttgttgatctacgacacttccaagttggcttccggtgttccatctagattctctggttccggttctggtactgagttcactttgactatctcttccttgcaaccagatgacttcgctacttactactgtttccagggttctggttacccattcactttcggtggtggtactaagttggagatcaagagaactgttgctgctccatccgttttcattttcccaccatccgacgaacaattgaagtccggtaccgcttccgttgtttgtttgttgaacaacttctacccacgtgaggctaaggttcagtggaaggttgacaacgctttgcaatccggtaactcccaagaatccgttactgagcaggattctaaggattccacttactcattgtcctccactttgactttgtccaaggctgattacgagaagcacaaggtttacgcttgcgaggttacacatcagggtttgtcctccccagttactaagtccttcaacagaggagagtgttaatag

SEQ ID NO: 37 - protein, 232 aa, Artificial Sequence
  Antibody light chain
MRFPSIFTAVLFAASSALADIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - DNA, 1410 bp, Artificial Sequence
  Antibody heavy chain
atgagattcccatccatcttcactgctgttttgttcgctgcttcttctgctttggctcaggttacattgagagaatccggtccagctttggttaagccaactcagactttgactttgacttgtactttctccggtttctccttgtctacttccggaatgtctgttggatggatcagacaaccacctggaaaggctttggaatggcttgctgacatttggtgggatgacaagaaggactacaacccatccttgaagtccagattgactatctccaaggacacttccaagaatcaagttgttttgaaggttacaaacatggacccagctgacactgctacttactactgtgctagatccatgatcactaactggtacttcgatgtttggggtgctggtactactgttactgtctcgagtgcttctactaagggaccatccgtttttccattggctccatcctctaagtctacttccggtggaaccgctgctttgggatgtttggttaaagactacttcccagagccagttactgtttcttggaactccggtgctttgacttctggtgttcacactttcccagctgttttgcaatcttccggtttgtactctttgtcctccgttgttactgttccatcctcttccttgggtactcagacttacatctgtaacgttaaccacaagccatccaacactaaggttgacaagagagttgagccaaagtcctgtgacaagacacatacttgtccaccatgtccagctccagaattgttgggtggtccatccgttttcttgttcccaccaaagccaaaggacactttgatgatctccagaactccagaggttacatgtgttgttgttgacgtttctcacgaggacccagaggttaagttcaactggtacgttgacggtgttgaagttcacaacgctaagactaagccaagagaagagcagtacaactccacttacagagttgtttccgttttgactgttttgcaccaggactggttgaacggtaaagaatacaagtgtaaggtttccaacaaggctttgccagctccaatcgaaaagactatctccaaggctaagggtcaaccaagagagccacaggtttacactttgccaccatccagagaagagatgactaagaaccaggtttccttgacttgtttggttaaaggattctacccatccgacattgctgttgagtgggaatctaacggtcaaccagagaacaactacaagactactccaccagttttggattctgatggttccttcttcttgtactccaagttgactgttgacaagtccagatggcaacagggtaacgttttctcctgttccgttatgcatgaggctttgcacaaccactacactcaaaagtccttgtctttgtcccctggttaatga

SEQ ID NO: 39 - protein, 468 aa, Artificial Sequence
  Antibody heavy chain
MRFPSIFTAVLFAASSALAQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - DNA, 705 bp, Artificial Sequence
  Antibody light chain
atgagattcccatccatcttcactgctgttttgttcgctgcttcttctgctttggctgacatccaaatgactcaatccccatcttctttgtctgcttccgttggtgacagagttactatcacttgtagagcttcccagggtatcagaaactacttggcttggtatcaacagaagccaggaaaggctccaaagttgttgatctacgctgcttccactttgcaatctggtgttccatctagattctctggttccggttctggtactgacttcactttgactatctcttccttgcaaccagaggacgttgctacttactactgtcagagatacaacagagcaccatacactttcggacagggtactaaggttgagatcaagagaactgttgctgctccatccgttttcattttcccaccatccgacgaacaattgaagtccggtaccgcttccgttgtttgtttgttgaacaacttctacccacgtgaggctaaggttcagtggaaggttgacaacgctttgcaatccggtaactcccaagaatccgttactgagcaggattctaaggattccacttactcattgtcctccactttgactttgtccaaggctgattacgagaagcacaaggtttacgcttgcgaggttacacatcagggtttgtcctccccagttactaagtccttcaacagaggagagtgttaatag

SEQ ID NO: 41 - protein, 233 aa, Artificial Sequence
  Antibody light chain
MRFPSIFTAVLFAASSALADIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - DNA, 1413 bp, Artificial Sequence
  Antibody heavy chain
atgagattcccatccatcttcactgctgttttgttcgctgcttcttctgctttggctgaggttcaattggttgaatccggtggtggattggttcaacctggtagatccttgagattgtcctgtgctgcttccggttttactttcgacgactacgctatgcattgggttagacaggctccaggtaaaggattggaatgggtttccgctattacttggaactccggtcacattgattacgctgactccgttgagggaagattcactatctccagagacaacgctaagaactccttgtacttgcagatgaactccttgagagctgaggatactgctgtttactactgtgctaaggtttcctacttgtccactgcttcttccttggattactggggacagggaactttggttactgtctcgagtgcttctactaagggaccatccgtttttccattggctccatcctctaagtctacttccggtggaaccgctgctttgggatgtttggttaaagactacttcccagagccagttactgtttcttggaactccggtgctttgacttctggtgttcacactttcccagctgttttgcaatcttccggtttgtactctttgtcctccgttgttactgttccatcctcttccttgggtactcagacttacatctgtaacgttaaccacaagccatccaacactaaggttgacaagaaggttgagccaaagtcctgtgacaagactcatacttgtccaccatgtccagctccagaattgttgggtggtccttccgtttttttgttcccaccaaagccaaaggacactttgatgatctccagaactccagaggttacatgtgttgttgttgacgtttctcacgaggacccagaggttaagttcaactggtacgttgacggtgttgaagttcacaacgctaagactaagccaagagaggagcagtacaactccacttacagagttgtttccgttttgactgttttgcaccaggattggttgaacggaaaggagtacaagtgtaaggtttccaacaaggctttgccagctccaatcgaaaagactatctccaaggctaagggtcaaccaagagagccacaggtttacactttgccaccatccagagatgagttaactaagaaccaggtttccttgacttgtttggttaagggattctacccatccgacattgctgttgaatgggagtctaacggtcaaccagagaacaactacaagactactccacctgttttggactctgacggttcctttttcttgtactccaagttgactgttgacaagtccagatggcaacagggtaacgttttctcctgttccgttatgcatgaggctttgcacaaccactacactcaaaagtccttgtctttgtcccctggttaatga

SEQ ID NO: 43 - protein, 469 aa, Artificial Sequence
  Antibody heavy chain
MRFPSIFTAVLFAASSALAEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - DNA, 705 bp, Artificial Sequence
  Antibody light chain
atgagattcccatccatcttcactgctgttttgttcgctgcttcttctgctttggctgacatccaaatgacacaatccccatcttccttgtctgcttccgttggtgacagagttactatcacttgttccgcttcccaagacatctccaactacttgaactggtatcagcagaagccaggtaaagctccaaaggttttgatctacttcacttcttccttgcactctggtgttccatctagattctctggttccggttctggtactgacttcactttgactatctcttccttgcaaccagaggacttcgctacttactactgtcagcagtactctactgttccatggactttcggacagggtactaaggttgagatcaagagaactgttgctgctccatccgttttcattttcccaccatccgacgaacaattgaagtccggtaccgcttccgttgtttgtttgttgaacaacttctacccacgtgaggctaaggttcagtggaaggttgacaacgctttgcaatccggtaactcccaagaatccgttactgagcaggattctaaggattccacttactcattgtcctccactttgactttgtccaaggctgattacgagaagcacaaggtttacgcttgcgaggttacacatcagggtttgtcctccccagttactaagtccttcaacagaggagagtgttaatag

SEQ ID NO: 45 - protein, 233 aa, Artificial Sequence
  Antibody light chain
MRFPSIFTAVLFAASSALADIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - DNA, 1419 bp, Artificial Sequence
  Antibody heavy chain
atgagattcccatccatcttcactgctgttttgttcgctgcttcttctgctttggctgaggttcagttggttgaatctggtggtggattggttcaacctggtggttctttgagattgtcctgtgctgcttccggttacactttcactaactacggaatgaactgggttagacaggctccaggtaaaggattggaatgggttggatggatcaacacttacactggtgaaccaacttacgctgctgacttcaagagaagattcactttttccttggacacttccaagtccactgcttacttgcagatgaactccttgagagctgaggatactgctgtttactactgtgctaagtacccacactactacggttcttcccactggtacttcgatgtttggggacagggaactttggttactgtctcgagtgcttctactaagggaccatccgtttttccattggctccatcctctaagtctacttccggtggaaccgctgctttgggatgtttggttaaagactacttcccagagccagttactgtttcttggaactccggtgctttgacttctggtgttcacactttcccagctgttttgcaatcttccggtttgtactctttgtcctccgttgttactgttccatcctcttccttgggtactcagacttacatctgtaacgttaaccacaagccatccaacactaaggttgacaagaaggttgagccaaagtcctgtgacaagacacatacttgtccaccatgtccagctccagaattgttgggtggtccatccgttttcttgttcccaccaaagccaaaggacactttgatgatctccagaactccagaggttacatgtgttgttgttgacgtttctcacgaggacccagaggttaagttcaactggtacgttgacggtgttgaagttcacaacgctaagactaagccaagagaagagcagtacaactccacttacagagttgtttccgttttgactgttttgcaccaggactggttgaacggtaaagaatacaagtgtaaggtttccaacaaggctttgccagctccaatcgaaaagactatctccaaggctaagggtcaaccaagagagccacaggtttacactttgccaccatccagagaagagatgactaagaaccaggtttccttgacttgtttggttaaaggattctacccatccgacattgctgttgagtgggaatctaacggtcaaccagagaacaactacaagactactccaccagttttggattctgatggttccttcttcttgtactccaagttgactgttgacaagtccagatggcaacagggtaacgttttctcctgttccgttatgcatgaggctttgcacaaccactacactcaaaagtccttgtctttgtcccctggttaatga

SEQ ID NO: 47 - protein, 471 aa, Artificial Sequence
  Antibody heavy chain
MRFPSIFTAVLFAASSALAEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2012/0135461 A1
(43) Pub. Date: May 31, 2012

(54) PRODUCTION OF GLYCOPROTEINS WITH REDUCED O-GLYCOSYLATION COMPRISING THE USE OF AN ALPHA-1,2-MANNOSIDASE

(76) Inventors: WILLIAM JAMES COOK, HANOVER, NH (US); STEFAN WILDT, LEBANON, NH (US)

(21) Appl. No.: 13/191,811

(22) Filed: Jul. 27, 2011

Related U.S. Application Data
(60) Provisional application No. 61/369,157, filed on Jul. 30, 2010.

(51) Int. Cl. C12P 21/00

(52) U.S. Cl. 435/68.1

(57) ABSTRACT

A method is described for producing protein compositions having reduced amounts of O-linked glycosylation. The method includes producing the protein in cells cultured in the presence of one or more α-1,2-mannosidases from Coccidiodes immitis, Coccidiodes posadasii, Penicillium citrinum, Magnaporthe grisea, Aspergillus saitoi, Aspergillus oryzae, and Chaetomiun globosum or a catalytically active fragment of said α-1,2-mannosidase.

### FIELD OF THE INVENTION

The present invention relates to compositions and methods for producing proteins having specific glycosylation patterns. In particular, the present invention relates to compositions and methods for producing proteins having reduced O-linked glycosylation.

### BACKGROUND OF THE INVENTION

Glycoproteins mediate many essential functions in humans and other mammals, including catalysis, signaling, cell-cell communication, and molecular recognition and association. Glycoproteins make up the majority of non-cytosolic proteins in eukaryotic organisms (Lis and Sharon, 1993, Eur. J. Biochem. 218:1-27). Many glycoproteins have been exploited for therapeutic purposes, and during the last two decades, recombinant versions of naturally-occurring glycoproteins have been a major part of the biotechnology industry. Variations in glycosylation patterns of recombinantly produced glycoproteins have recently been the topic of much attention in the scientific community as recombinant proteins produced as potential prophylactics and therapeutics approach the clinic.

In general, the glycosylation structures of glycoprotein oligosaccharides will vary depending upon the host species of the cells used to produce them. Therapeutic proteins produced in non-human host cells are likely to contain non-human glycosylation which may elicit an immunogenic response in humans—e.g. hypermannosylation in yeast (Ballou, 1990, Methods Enzymol. 185:440-470); α(1,3)-fucose and β(1,2)-xylose in plants, (Cabanes-Macheteau et al., 1999. Glycobiology, 9: 365-372); N-glycolylneuraminic acid in Chinese hamster ovary cells (Noguchi et al., 1995. J. Biochem. 117: 5-62); and, Galα-1,3Gal glycosylation in mice (Borrebaeck, et al., 1993, Immun. Today, 14: 477-479). Carbohydrate chains bound to proteins in animal cells include N-glycoside bond type carbohydrate chains (also called N-glycans; or N-linked glycosylation) bound to an asparagine (Asn) residue in the protein and O-glycoside bond type carbohydrate chains (also called O-glycans; or O-linked glycosylation) bound to a serine (Ser) or threonine (Thr) residue in the protein.

Because the oligosaccharide structures of glycoproteins produced by non-human mammalian cells tend to be more closely related to those of human glycoproteins, most commercial glycoproteins are produced in mammalian cells. However, mammalian cells have several important disadvantages as host cells for protein production. Besides being costly, processes for producing proteins in mammalian cells produce heterogeneous populations of glycoforms, have low volumetric titers, and require both ongoing viral containment and significant time to generate stable cell lines.

It is well recognized that the particular glycoforms on a protein can profoundly affect the properties of the protein, including its pharmacokinetic, pharmacodynamic, receptor-interaction, and tissue-specific targeting properties (Graddis et al., 2002, Curr Pharm Biotechnol. 3: 285-297). For example, it has been shown that different glycosylation patterns of Igs are associated with different biological properties (Jefferis and Lund, 1997, Antibody Eng. Chem. Immunol., 65: 111-128; Wright and Morrison, 1997, Trends Biotechnol., 15: 26-32). It has further been shown that galactosylation of a glycoprotein can vary with cell culture conditions, which may render some glycoprotein compositions immunogenic depending on the specific galactose pattern on the glycoprotein (Patel et at, 1992, Biochem J. 285: 839-845). However, because it is not known which specific glycoform(s) contribute(s) to a desired biological function, the ability to enrich for specific glycoforms on glycoproteins is highly desirable. Because different glycoforms are associated with different biological properties, the ability to enrich for glycoproteins having a specific glycoform can be used to elucidate the relationship between a specific glycoform and a specific biological function of the glycoprotein. Also, the ability to enrich for glycoproteins having a specific glycoform enables the production of therapeutic glycoproteins having particular specificities. Thus, production of glycoprotein compositions that are enriched for particular glycoforms is highly desirable.

While the pathway for N-linked glycosylation has been the subject of much analysis, the process and function of O-linked glycosylation is not as well understood. However, it is known that O-glycosylation is a posttranslational event, which occurs in the cis-Golgi (Varki, 1993, Glycobiol., 3: 97-130). While a consensus acceptor sequence for O-linked glycosylation like that for N-linked glycosylation does not appear to exist, a comparison of amino acid sequences around a large number of O-linked glycosylation sites of several glycoproteins show an increased frequency of proline residues at positions −1 and +3 relative to the glycosylated residues and a marked increase of serine, threonine, and alanine residues (Wilson et al., 1991, Biochem. J., 275: 529-534). Stretches of serine and threonine residues in glycoproteins, may also be potential sites for O-glycosylation.

One gene family that has a role in O-linked glycosylation are the genes encoding the Dol-P-Man:Protein (Ser/Thr) Mannosyl Transferase (Pmt). These highly conserved genes have been identified in both higher eukaryotes such as humans, rodents, insects, and the like and lower eukaryotes such as fungi and the like. International Patent Publication No. WO2007/061631, the contents of which are hereby incorporated by reference, discloses certain methods of reducing β-glycosylation, particularly in fungi and yeast cells using particular chemical compounds, alone or in combination with certain α-1,2-mannosidases. However, there is still a need for further methods of reducing O-glycosylation using other α-1,2-mannosidases.

### BRIEF SUMMARY OF THE INVENTION

The present invention provides methods for producing glycoproteins having specific glycosylation patterns. In particular, the present invention provides a method for making a glycoprotein in a host cell in which the O-linked glycosylation of the glycoprotein is reduced by contacting the host cells with one or more α-1,2-mannosidases from Coccidiodes immitis, Coccidiodes posadasii, Penicillium citrinum, Magnaporthe grisea, Aspergillus saitoi, Aspergillus oryzae, and Chaetomiun globosum or a catalytically active fragment of said α-1,2-mannosidase.

In one aspect, the invention provides a method of producing a glycoprotein having reduced O-linked glycosylation comprising: (a) providing a nucleic acid encoding a glycoprotein; (b) introducing the nucleic acid into a host cell; (c) contacting the host cell with an α-1,2-mannosidase enzyme, wherein the α-1,2-mannosidase enzyme comprises an amino acid sequence selected from the group consisting of: SEQ ID NO: 4 (Coccidiodes immitis), SEQ ID NO: 6 (Coccidiodes posadasii), SEQ ID NO: 8 (Penicillium citrinum), SEQ ID NO: 10 (Magnaporthe grisea), SEQ ID NO: 12 (Aspergillus saitoi), SEQ ID NO: 14 (Aspergillus oryzae), and SEQ ID NO: 16 (Chaetomiun globosum) (or a catalytically active fragment or variant of said sequence) and (d) isolating the glycoprotein produced by the host cell in the presence of the α-1,2-mannosidase enzyme; thereby producing a glycoprotein having reduced O-linked glycosylation. In preferred aspects of the invention, the α-1,2-mannosidase enzyme comprises SEQ ID NO: 4 (Coccidiodes immitis). In other preferred aspects of the invention, the α-1,2-mannosidase enzyme comprises SEQ ID NO: 6 (Coccidiodes posadasii).

In one embodiment; the host cell is grown for a time sufficient to provide a multiplicity of the host cells having the nucleic acid encoding the glycoprotein before contacting the host cell with the α-1,2-mannosidase enzyme. In another embodiment, the host cell is grown in the presence of the α-1,2-mannosidase enzyme.

In one embodiment, the nucleic acid encoding the glycoprotein is operably linked to an inducible promoter. In another embodiment, the nucleic acid encoding the glycoprotein is operably linked to a constitutive promoter. In certain embodiments of the invention where the nucleic acid encoding the glycoprotein is operably linked to an inducible promoter, the host cell is contacted with an inducer of the promoter to induce expression of the glycoprotein for a time before contacting the host cell with the α-1,2-mannosidase enzyme.

While the method can be performed using any host cell that produce glycoproteins having O-linked glycosylation, in currently preferred aspects, the host cell is a lower eukaryotic cell. A lower eukaryotic host cell when used herein in connection with glycosylation profiles, refers to any eukaryotic cell which ordinarily produces high mannose containing N-linked glycans, and thus, includes most typical lower eukaryotic cells, including uni- and multi-cellular fungal and algal cells. In preferred embodiments, the host cell is a fungal cell or a yeast cell. In even preferred embodiments, the host cell is selected from the group consisting of K. lactis, Pichia pastoris, Pichia methanolica, and Hansenula polymorpha. In one embodiment, the host cell is Pichia pastoris. In some embodiments, the host cell is a yeast or filamentous fungal cell that has been genetically modified to produce glycoproteins with a predominant N-glycan glycoform. In some embodiments, the host cell has been genetically modified to produce glycoproteins in which the N-glycosylation pattern is human-like or humanized.

In certain embodiments of the claimed method, the glycoprotein is produced at a yield of at least 100 mg/liter of culture medium, preferably at a yield of at least 1 g/liter of culture medium, and more preferably at a yield of at least 3 g/liter of culture medium.

In other embodiments, the claimed method further comprises contacting the host cell with one or more inhibitors of Pmt-mediated O-linked glycosylation. In some embodiments, the host cell is grown for a time sufficient to provide a multiplicity of the host cells having the nucleic acid encoding the glycoprotein before contacting the host cell with the one or more inhibitors of Pmt-mediated O-linked glycosylation. In other embodiments, the host cell is grown in the presence of the one or more inhibitors of Pmt-mediated O-linked glycosylation. In certain embodiments, the inhibitor of Pmt-mediated glycosylation is a benzylidene thiazolidinedione. In other embodiments, the one or more inhibitors of Pmt-mediated O-linked glycosylation are selected from the group consisting of: 5-[[3,4-bis(phenylmethoxy) phenyl]methylene]-4-oxo-2-thioxo-3-thiazolidineacetic Acid; 5-[[3-(1-Phenylethoxy)-4-(2-phenylethoxy)]phenyl]methylene]-4-oxo-2-thioxo-3-thiazolidineacetic Acid; and 5-[[3-(1-Phenyl-2-hydroxy)ethoxy)-4-(2-phenylethoxy)]phenyl]methylene]-4-oxo-2-thioxo-3-thiazolidineacetic Acid. In other embodiments, the one or more inhibitors of Pmt-mediated O-linked glycosylation are selected from the group of inhibitors disclosed and claimed in International Publication No. WO2009/143041. In one embodiment, the one or more inhibitors of Pmt-mediated O-linked glycosylation are selected from the group consisting of:

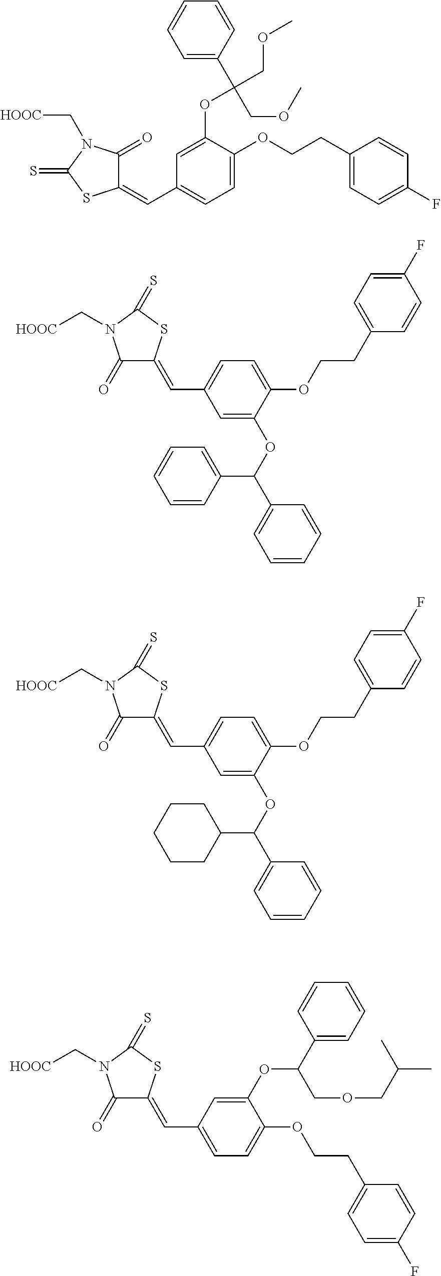

or a salt thereof. In one embodiment, the one or more inhibitor of Pmt-mediated O-linked glycosylation is:

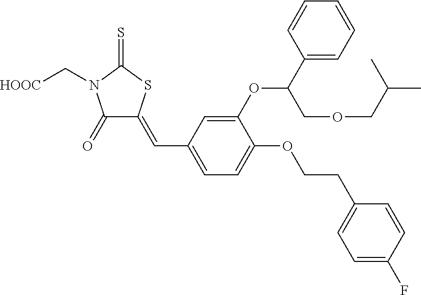

or a salt thereof.

In another aspect, the invention provides a method of producing a glycoprotein having reduced O-linked glycosylation comprising: (a) providing a first nucleic acid encoding a glycoprotein; (b) providing a second nucleic acid encoding an α-1,2-mannosidase enzyme, wherein the α-1,2-mannosidase enzyme comprises an amino acid sequence selected from the group consisting of: SEQ ID NO: 4 (Coccidiodes immitis), SEQ ID NO: 6 (Coccidiodes posadasii), SEQ ID NO: 8 (Penicillium citrinum), SEQ ID NO: 10 (Magnaporthe grisea), SEQ ID NO: 12 (Aspergillus saitoi), SEQ ID NO: 14 (Aspergillus oryzae), and SEQ ID NO: 16 (Chaetomiun globosum) (or a catalytically active fragment or variant of said sequence); (c) introducing the first and second nucleic acid into a host cell; (d) expressing the first and second nucleic acids in the host cell; and (e) isolating the glycoprotein produced by the host cell in the presence of the α-1,2-mannosidase enzyme, thereby producing a glycoprotein having reduced O-linked glycosylation. In preferred aspects of the invention, the α-1,2-mannosidase enzyme comprises SEQ ID NO: 4 (Coccidiodes immitis). In other preferred aspects of the invention, the α-1,2-mannosidase enzyme comprises SEQ ID NO: 6 (Coccidiodes posadasii).

In one embodiment, the host cell is grown for a time sufficient to provide a multiplicity of the host cells having the nucleic acid encoding the glycoprotein before contacting the host cell with the α-1,2-mannosidase enzyme. In another embodiment, the host cell is grown in the presence of the α-1,2-mannosidase enzyme.

In one embodiment, first nucleic acid encoding the glycoprotein is operably linked to an inducible promoter. In another embodiment, the second nucleic acid encoding the α-1,2-mannosidase enzyme is operably linked to an inducible promoter. In yet other embodiments, the first and the second nucleic acids are operably linked to inducible promoters.

In certain embodiments of the invention where the nucleic acids encoding the glycoprotein and the α-1,2-mannosidase enzyme are operably linked to an inducible promoter, the host cell is contacted with an inducer of the promoter to induce expression of the glycoprotein for a time before inducing expression of the α-1,2-mannosidase enzyme. In other embodiments of the invention where the nucleic acids encoding the glycoprotein and the α-1,2-mannosidase enzyme are operably linked to an inducible promoter, the host cell is induced to express the α-1,2-mannosidase enzyme before inducing expression of the glycoprotein.

While the method can be performed using any host cell that produced proteins having O-linked glycosylation, in currently preferred aspects, the host cell is a lower eukaryotic cell. In preferred embodiments, the host cell is a fungal cell or a yeast cell. In even preferred embodiments, the host cell is selected from the group consisting of K. lactis, Pichia pastoris, Pichia methanolica, and Hansenula polymorpha. In one embodiment, the host cell is Pichia pastoris. In some embodiments, the host cell is a yeast or filamentous fungal cell that has been genetically modified to produce glycoproteins with a predominant N-glycan glycoform. In some embodiments, the host cell has been genetically modified to produce glycoproteins in which the N-glycosylation pattern is human-like or humanized.

In certain embodiments of the claimed method, the glycoprotein is produced at a yield of at least 100 mg/liter of culture medium, preferably at a yield of at least 1 g/liter of culture medium, and more preferably at a yield of at least 3 g/liter of culture medium.

In other embodiments, the claimed method further comprises contacting the host cell with one or more inhibitors of Pmt-mediated O-linked glycosylation. In some embodiments, the host cell is grown for a time sufficient to provide a multiplicity of the host cells having the nucleic acid encoding the glycoprotein before contacting the host cell with the one or more inhibitors of Pmt-mediated O-linked glycosylation. In other embodiments, the host cell is grown in the presence of the one or more inhibitors of Pmt-mediated O-linked glycosylation. In certain embodiments, the inhibitor of Pmt-mediated O-linked glycosylation is a benzylidene thiazolidinedione. In other embodiments, the one or more inhibitors of Pmt-mediated O-linked glycosylation are selected from the group consisting of: 5-[[3,4-bis(phenylmethoxy) phenyl]methylene]-4-oxo-2-thioxo-3-thiazolidineacetic Acid; 5-[[3-(1-Phenylethoxy)-4-(2-phenylethoxy)]phenyl]methylene]-4-oxo-2-thioxo-3-thiazolidineacetic Acid; and 5-[[3-(1-Phenyl-2-hydroxy)ethoxy)-4-(2-phenylethoxy)]phenyl]methylene]-4-oxo-2-thioxo-3-thiazolidineacetic Acid. In other embodiments, the one or more inhibitors of Pmt-mediated (-linked glycosylation are selected from the group of inhibitors disclosed and claimed in International Publication No. WO2009/143041. In one embodiment, the one or more inhibitors of Pmt-mediated O-linked glycosylation are selected from the group consisting of:

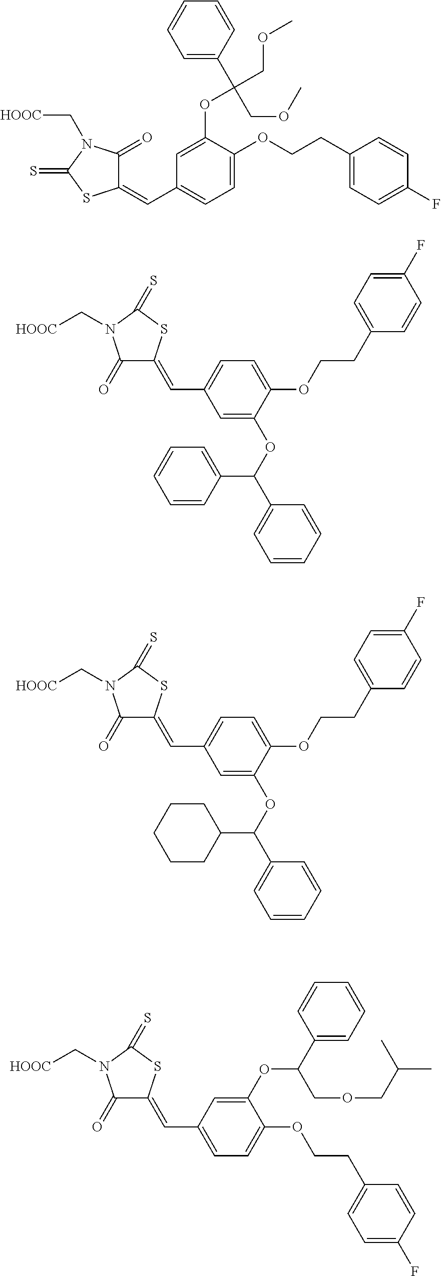

or a salt thereof. In one embodiment, the one or more inhibitor of Pmt-mediated O-linked glycosylation is:

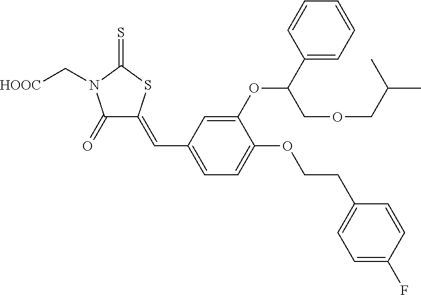

or a salt thereof.

In certain preferred embodiments, the α-1,2-mannosidase may be produced from a chimeric construct comprising a nucleic acid sequence encoding the catalytic domain of an α-1,2-mannosidase selected from the group consisting of Coccidiodes immitis, Coccidiodes posadasii, Penicillium citrinum, Magnaporthe grisea, Aspergillus saitoi, Aspergillus oryzae, and Chaetomiun globosum operatively linked to a nucleic acid sequence encoding a cellular targeting signal peptide not normally associated with the catalytic domain. In other embodiments, the α-1,2-mannosidase may be separately produced and added to the cell culture, or may be produced by co-expressing the α-1,2-mannosidase with the recombinant glycoprotein.

Pmt-mediated O-linked glycosylation refers to O-linked glycosylation wherein transfer of mannose residues to the serine or threonine residues of a protein is mediated by a protein-O-D-mannosyltransferase (Pmt) or homologue encoded by a PMT gene or its homologue. The inhibitors of Pmt-mediated O-linked glycosylation include inhibitors that inhibit any one of the homologues of the PMT genes. In a currently preferred aspect, the inhibitor inhibits at least Pmt1 and/or Pmt2 activity of fungi and yeast, or the corresponding homologue in other organisms, including but not limited to, mammals, plants, and insects.

All publications, patents, patent applications, and other references mentioned herein are hereby incorporated by reference in their entireties.

### BRIEF DESCRIPTION OF THE DRAWINGS

FIG. 1 illustrates the effect of heterologous α-1,2-mannosidase expression on O-glycosylation of secreted recombinant monoclonal antibody (anti-HER2) in P. pastoris. Western blotting using an anti-human IgG (H+L) antibody was used to detect the heavy (Hc) chain in the growth media of strains co-expressing α-Her2 antibody and heterologous α-1,2-mannosidases. The slowest migrating band is the β-glycosylated Hc whose migration is increased due to reduction of O-mannose chain length in strains co-expressing A. oryzae (AoMNS, Panel A), C. immitis (CiMNS, Panel A) or C. posadasii (CpMNS, Panel B), but not P. nodorum (PnMNS, Panel B) α-1,2-mannosidase.

### DETAILED DESCRIPTION OF THE INVENTION

The present invention provides a method for expressing a recombinant glycoprotein, which is susceptible to O-linked glycosylation in a particular host cell, having a reduced amount of O-linked glycosylation (including no O-linked glycosylation) in that cell type. The method involves inducing expression of a glycoprotein of interest in a host cell in which the protein is susceptible to O-linked glycosylation in the host cell in the presence of one or more α-1,2-mannosidases from Coccidiodes immitis, Coccidiodes posadasii, Penicillium citrinum, Magnaporthe grisea, Aspergillus saitoi, Aspergillus oryzae, and Chaetomiun globosum or a catalytically active fragment of any of said α-1,2-mannosidases. The glycoprotein that is expressed in the presence of the one or more α-1,2-mannosidases has a reduced amount of O-linked glycosylation compared to the amount of O-linked glycosylation that would have been present on the glycoprotein if it had been produced in the absence of one or more α-1,2-mannosidases.

The method is an improvement over prior art methods for producing glycoproteins having reduced O-linked glycosylation in host cells in which the proteins are susceptible to O-linked glycosylation. This improvement facilitates the production of glycoproteins having reduced O-linked glycosylation in host cells that have been genetically modified to produce glycoproteins having predominantly a particular N-linked glycan structure but which also O-glycosylate the glycoprotein. Methods for producing a wide variety of glycoproteins having predominantly particular N-linked glycoforms have been disclosed in U.S. Pat. Nos. 7,029,872, 7,449,308, 7,465,577, 7,259,007, 7,625,756, 7,598,055 and 7,332,299 and U.S. Published Application Nos. 20050170452, 20050260729, and 20060040353. Any one of the host cells described in the aforementioned patent and patent applications can be used to produce a glycoprotein having predominantly a particular N-linked glycan structure and having reduced O-linked glycosylation using the method disclosed herein. It has been found that some host cells that have been genetically modified to produce glycoproteins having predominantly a particular N-linked glycan structure can grow less well in culture under particular conditions than host cells that have not been modified. For example, particular fungal and yeast cells in which genes involved in hypermannosylation have been deleted and other genes needed to produce particular mammalian or human like N-linked glycan structures have been added, can grow less well than fungal or yeast cells that do not contain the genetic modifications. In some of these genetically modified fungal or yeast cells, further introducing deletions of the PMT1 or PMT2 genes either is lethal to the cells or adversely affects the ability of the cells to grow to sufficient quantities in culture. The method herein avoids the potential deleterious effects of deleting the PMT1 and PMT2 genes by allowing the cells to grow to sufficient quantities in culture before inducing expression of the recombinant glycoprotein and adding an inhibitor of the activity of the Pmt proteins, or one or more α-1,2-mannosidases, or both, to produce the recombinant glycoprotein having predominantly particular N-linked glycan structures and reduced O-linked glycosylation.

Therefore, an important aspect of the method is that it provides for a glycoprotein composition comprising reduced O-linked glycosylation and predominantly a specific N-linked glycoform in which the recombinant glycoprotein may exhibit increased biological activity and/or decreased undesired immunogenicity relative to compositions of the same glycoprotein produced from mammalian cell culture, such as CHO cells. An additional advantage of producing the glycoprotein composition comprising reduced O-linked glycosylation and a predominant N-linked glycoform is that it avoids production of undesired or inactive glycoforms and heterogeneous mixtures, which may induce undesired effects and/or dilute the more effective glycoform. Thus, therapeutic pharmaceutical composition of glycoprotein molecules comprising, for example, predominantly an N-glycan selected from the group consisting of: Man₅GlcNAc₂, GlcNAcMan₅GlcNAc₂, GalGlcNAcMan₅GlcNAc₂, NANAGalGlcNAcMan₅GlcNAc₂, GlcNAcMan₃GlcNAc₂, GlcNAc₍₁₋₄₎Man₃GlcNAc₂, Gal₍₁₋₄₎GlcNAc₍₁₋₄₎Man₃GlcNAc₂, and NANA₍₁₋₄₎Gal₍₁₋₄₎GlcNAc₍₁₋₄₎Man₃GlcNAc₂, wherein the subscript indicates the number of the particular sugar residues on the N-glycan structure, and having reduced O-linked glycosylation may well be effective at lower doses, thus having higher efficacy/potency. Examples of N-glycan structures include but are not limited to Man₅GlcNAc₂, GlcNAcMan₅GlcNAc₂, GlcNAcMan₃GlcNAc₂, GlcNAc₂Man₃GlcNAc₂, GlcNAc₃Man₃GlcNAc₂, GlcNAc₄Man₃GlcNAc₂, GalGlcNAc₂Man₃GlcNAc₂, Gal₂GlcNAc₂Man₃GlcNAc₂, Gal₂GlcNAc₃Man₃GlcNAc₂, Gal₂GlcNAc₄Man₃GlcNAc₂, Gal₃GlcNAc₃Man₃GlcNAc₂, Gal₃GlcNAc₄Man₃GlcNAc₂, Gal₄GlcNAc₄Man₃GlcNAc₂, NANAGal₂GlcNAc₂Man₃GlcNAc₂, NANA₂Gal₂GlcNAc₂Man₃GlcNAc₂, NANA₃Gal₃GlcNAc₃Man₃GlcNAc₂, and NANA₄Gal₄GlcNAc₄Man₃GlcNAc₂.

In general, the method for producing proteins having reduced O-linked glycosylation comprises transforming a host cell with a nucleic acid encoding a recombinant or heterologous protein in which it is desirable to produce the protein having reduced O-linked glycosylation. The nucleic acid encoding the recombinant protein is operably linked to regulatory sequences that allow expression of the recombinant protein. Such regulatory sequences include an inducible promoter and optionally an enhancer upstream, or 5′, to the nucleic acid encoding the fusion protein and a transcription termination site 3′ or down stream from the nucleic acid encoding the recombinant protein. The nucleic acid also typically encodes a 5′ UTR region having a ribosome binding site and a 3′ untranslated region. The nucleic acid is often a component of a vector replicable in cells in which the recombinant protein is expressed. The vector can also contain a marker to allow recognition of transformed cells. However, some cell types, particularly yeast, can be successfully transformed with a nucleic acid lacking extraneous vector sequences.

Nucleic acids encoding desired recombinant proteins can be obtained from several sources. cDNA sequences can be amplified from cell lines known to express the protein using primers to conserved regions (see, for example, Marks et al., J. Mol. Biol. 581-596 (1991)). Nucleic acids can also be synthesized de novo based on sequences in the scientific literature. Nucleic acids can also be synthesized by extension of overlapping oligonucleotides spanning a desired sequence (see, e.g., Caldas et al., Protein Engineering, 13, 353-360 (2000)).

In one aspect, the nucleic acid encoding the protein is operably linked to an inducible promoter, which allows expression of the protein to be induced when desired. In another aspect, the nucleic acid encoding the protein is operably linked to a constitutive promoter. To facilitate isolation of the expressed protein, it is currently preferable that the protein include a signal sequence that directs the protein to be excreted into the cell culture medium where it can then be isolated. In the first aspect, the transformed host cells are cultured for a time sufficient to produce a desired multiplicity of host cells sufficient to produce the desired amount of protein before adding one or more inhibitors of Pmt-mediated O-linked glycosylation to the culture medium. The inducer and inhibitor can be added to the culture simultaneously or the inducer is added to the culture before adding the one or more Pmt inhibitors or the one or more Pmt inhibitors is added to the culture before adding the inducer. The induced protein is produced having reduced O-linked glycosylation and can be recovered from the culture medium or for proteins not having a signal sequence, from the host cell by lysis. In the second aspect, wherein the nucleic acid encoding the protein is operably linked to a constitutive promoter, the one or more inhibitors of Pmt-mediated O-linked glycosylation is added to the culture medium at the same time the culture is established and the protein, which is produced having reduced O-linked glycosylation, can be recovered from the culture medium or for proteins not having a signal sequence, from the host cell by lysis. Inhibitors useful for producing proteins with reduced O-linked glycosylation are chemicals or compositions that inhibit the activity one or more of the Pmt proteins. When the host cell is a lower eukaryote such as fungi or yeast, it is desirable that the inhibitor inhibit at least the activity of Pmt1 or Pmt2, or both. In higher eukaryotes, it is desirable that the inhibitor inhibit activity of the homologue in the higher eukaryote that corresponds to the Pmt1 or Pmt2. Chemical inhibitors that can be used include the benzylidene thiazolidinediones identified in U.S. Pat. No. 7,105,554, WO2007/061631 or WO2009/143041.

The α-1,2-mannosidase can be produced from a chimeric nucleic acid comprising a nucleic acid sequence encoding at least the catalytic domain of an α-1,2-mannosidase, which is capable of trimming multiple mannose residues from O-linked glycans, operatively linked to a nucleic acid sequence encoding a cellular targeting signal peptide not normally associated with the catalytic domain. The chimeric nucleic acid can be operably linked to a constitutive or inducible promoter. The chimeric nucleic acid is transformed into a host cell to produce the α-1,2-mannosidase, which is then isolated and then added to the cell culture medium containing cells transformed with the nucleic acid encoding the heterologous protein at the time expression of the protein is induced. Alternatively, the host cell is transformed with the chimeric nucleic acid encoding the α-1,2-mannosidase and the nucleic acid encoding the recombinant protein and co-expressing the α-1,2-mannosidase and the recombinant protein at the same time. In particular embodiments, both the chimeric nucleic acid encoding the α-1,2-mannosidase and the nucleic acid encoding the recombinant protein are both operably linked to an inducible promoter. In other embodiments, one or both of the promoters are constitutive.

In one aspect, the nucleic acid encoding the recombinant protein is operably linked to an inducible promoter, which allows expression of the recombinant protein to be induced when desired. In another aspect, the nucleic acid encoding the protein is operably linked to a constitutive promoter. To facilitate isolation of the expressed recombinant protein, it is preferable that the protein include a signal sequence that directs the recombinant protein to be excreted into the cell culture medium where it can then be isolated.

In the first aspect, the transformed host cells are cultured for a time sufficient to produce a desired multiplicity of host cells sufficient to produce the desired amount of the recombinant protein before adding the one or more α-1,2-mannosidases to the culture medium. The inducer and the one or more α-1,2-mannosidases can be added to the culture simultaneously or the inducer is added to the culture before adding the one or more α-1,2-mannosidases or the one or more α-1,2-mannosidases is added to the culture before adding the inducer. The induced recombinant protein is produced having reduced O-linked glycosylation and can be recovered from the culture medium or for proteins not having a signal sequence, from the host cell by lysis.

In the second aspect, wherein the nucleic acid encoding the recombinant protein is operably linked to a constitutive promoter, the one or more α-1,2-mannosidases is added to the culture medium at the same time the culture is established and the recombinant protein, which is produced having reduced O-linked glycosylation, can be recovered from the culture medium or for recombinant proteins not having a signal sequence, from the host cell by lysis.

In a further still aspect for producing proteins having reduced O-linked glycosylation without using an inhibitor of Pmt-mediated O-linked glycosylation, the host cell is transformed with a chimeric nucleic acid encoding the α-1,2-mannosidase and a nucleic acid encoding the recombinant protein and co-expressing the α-1,2-mannosidase and the recombinant protein to produce the recombinant protein having reduced O-linked glycosylation. In particular embodiments, both the chimeric nucleic acid encoding the α-1,2-mannosidase and the nucleic acid encoding the recombinant proteins are both operably linked to an inducible promoter. In other embodiments, one or both of the promoters are constitutive. In the case of an inducible promoter, the host cells are grown to produce a desired multiplicity of host cells before inducing expression of the α-1,2-mannosidase and/or recombinant protein.

### II. Host Cells

While host cells for the method herein includes both higher eukaryote cells and lower eukaryote cells, lower eukaryote cells, for example filamentous fungi or yeast cells, are currently preferred for expression of proteins because they can be economically cultured, give high yields of protein, and when appropriately modified are capable of producing proteins having suitable glycosylation patterns. Lower eukaryotes include yeast, fungi, collar-flagellates, microsporidia, alveolates (e.g., dinoflagellates), stramenopiles (e.g, brown algae, protozoa), rhodophyta (e.g., red algae), plants (e.g., green algae, plant cells, moss) and other protists. Yeast and fungi include, but are not limited to: Pichia sp. (for example, Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica), Saccharomyces sp. (for example Saccharomyces cerevisaie), Hansenula polymorpha, Kluyveromyces sp. (for example, Kluyveromyces lactis), Candida albicans, Aspergillus sp (for example, Aspergillus nidulans, Aspergillus niger, Aspergillus oryzae), Trichoderma reesei, Chrysosporium lucknowense, Fusarium sp. (for example, Fusarium gramineum, Fusarium venenatum), Physcomitrella patens and Neurospora crassa. Yeast, in particular, are currently preferred because yeast offers established genetics allowing for rapid transformations, tested protein localization strategies, and facile gene knock-out techniques. Suitable vectors have expression control sequences, such as promoters, including 3-phosphoglycerate kinase or other glycolytic enzymes, and an origin of replication, termination sequences, and the like as desired.

Various yeasts, such as K. lactis, Pichia pastoris, Pichia methanolica, and Hansenula polymorpha are currently preferred for cell culture because they are able to grow to high cell densities and secrete large quantities of recombinant protein. Likewise, filamentous fungi, such as Aspergillus niger, Fusarium sp, Neurospora crass, and others can be used to produce recombinant proteins at an industrial scale.

Lower eukaryotes, in particular filamentous fungi and yeast, can be genetically modified so that they express proteins or glycoproteins in which the glycosylation pattern is human-like or humanized. This can be achieved by eliminating selected endogenous glycosylation enzymes and/or supplying exogenous enzymes as described by Gerngross et al. in U.S. Pat. Nos. 7,029,872, 7,449,308, 7,465,577, 7,259,007, 7,625,756, 7,598,055 and 7,332,299 and U.S. Published Application Nos. 20050170452, 20050260729, and 20060040353. Thus, a host cell can additionally or alternatively be engineered to express one or more enzymes or enzyme activities, which enable the production of particular N-glycan structures at a high yield. Such an enzyme can be targeted to a host subcellular organelle in which the enzyme will have optimal activity, for example, by means of signal peptide not normally associated with the enzyme. Host cells can also be modified to express a sugar nucleotide transporter and/or a nucleotide diphosphatase enzyme. The transporter and diphosphatase improve the efficiency of engineered glycosylation steps, by providing the appropriate substrates for the glycosylation enzymes in the appropriate compartments, reducing competitive product inhibition, and promoting the removal of nucleoside diphosphates. See, for example, Gerngross et al. in U.S. Pat. No. 7,449,308 and Hamilton, 2003, Science 301: 1244-46 and the aforementioned U.S. patent and patent applications.

By way of example, a host cell (for example, yeast or fungal) can be selected or engineered to be depleted in 1,6-mannosyl transferase activities, which would otherwise add mannose residues onto the N-glycan of a glycoprotein, and to further include a nucleic acid for ectopic expression of an α-1,2 mannosidase activity, which enables production of recombinant glycoproteins having greater than 30 mole percent Man₅GlcNAc₂ N-glycans. When a glycoprotein is produced in the host cells according to the method described herein, the host cells will produce a glycoprotein having predominantly a Man₅GlcNAc₂ N-glycan structure and reduced O-glycosylation compared to the glycoprotein produced in the cell otherwise. In a further aspect, the host cell is engineered to further include a nucleic acid for ectopic expression of GlcNAc transferase I activity, which enables production of glycoproteins having predominantly GlcNAcMan₅GlcNAc₂ N-glycans. When a glycoprotein is produced in the host cells according to the method described herein, the host cells will produce a glycoprotein having predominantly a GlcNAcMan₅GlcNAc₂ N-glycan structure and reduced O-glycosylation compared to the glycoprotein produced in the cell otherwise. In a further still aspect, the host cell is engineered to further include a nucleic acid for ectopic expression of mannosidase II activity, which enables production of glycoproteins having predominantly GlcNAcMan₃GlcNAc₂ N-glycans. When a glycoprotein is produced in the host cells according to the method described herein, the host cells will produce a glycoprotein having predominantly a GlcNAcMan₃GlcNAc₂ N-glycan structure and reduced O-glycosylation compared to the glycoprotein produced in the cell otherwise. In a further still aspect, the host cell is engineered to further include a nucleic acid for ectopic expression of GlcNAc transferase II activity, which enables production of glycoproteins having predominantly GlcNAc₂Man₃GlcNAc₂ N-glycans. When a glycoprotein is produced in the host cells according to the method described herein, the host cells will produce a glycoprotein having predominantly a GlcNAc₂Man₃GlcNAc₂ N-glycan structure and reduced O-glycosylation compared to the glycoprotein produced in the cell otherwise. In further still aspects, the above host cells can be further engineered to produce particular hybrid or complex N-glycan or human-like N-glycan structures by further including one or more higher eukaryote genes involved in N-linked glycosylation, in any combination, that encode for example, sialytransferase activities, class II and III mannosidase activities, GlcNAc transferase II, III, IV, V, VI, IX activity, and galactose transferase activity. It is currently preferable that the cells further include one or more of nucleic acids encoding UDP-specific diphosphatase activity, GDP-specific diphosphatase activity, and UDP-GlcNAc transporter activity.

Plants and plant cell cultures may be used for expression of proteins and glycoproteins with reduced O-linked glycosylation as taught herein (See, for example, Larrick & Fry, 1991, Hum. Antibodies Hybridomas 2: 172-89); Benvenuto et al., 1991, Plant Mol. Biol. 17: 865-74); Durin et al., 1990, Plant Mol. Biol. 15: 281-93); Hiatt et al., 1989, Nature 342: 76-8). Preferable plant hosts include, for example, Arabidopsis, Nicotiana tabacum, Nicotiana rustica, and Solanum tuberosum.

Insect cell culture can also be used to produce proteins and glycoproteins proteins and glycoproteins with reduced O-linked glycosylation, as taught herein for example, baculovirus-based expression systems (See, e.g., Putlitz et al., 1990, Bio/Technology 8: 651-654).

Although not currently as economical to culture as lower eukaryotes and prokaryotes, mammalian tissue cell culture can also be used to express and produce proteins and glycoproteins with reduced O-linked glycosylation as taught herein (See Winnacker, From Genes to Clones (VCH Publishers, N.Y., 1987). Suitable hosts include CHO cell lines, various COS cell lines, HeLa cells, preferably myeloma cell lines or the like or transformed B-cells or hybridomas. Expression vectors for these cells can include expression control sequences, such as an origin of replication, a promoter, an enhancer (Queen et al., 1986, Immunol. Rev. 89:49-68), and necessary processing information sites, such as ribosome binding sites, RNA splice sites, polyadenylation sites, and transcriptional terminator sequences. Expression control sequences are promoters derived from immunoglobulin genes, SV40, Adenovirus, bovine Papilloma Virus, cytomegalovirus and the like. Generally, a selectable marker, such as a neoR expression cassette, is included in the expression vector.

The nucleic acid encoding the protein to be expressed can be transferred into the host cell by conventional methods, which vary depending on the type of cellular host. For example, calcium phosphate treatment, protoplast fusion, natural breeding, lipofection, biolistics, viral-based transduction, or electroporation can be used for cellular hosts. Tungsten particle ballistic transgenesis is preferred for plant cells and tissues. (See, generally, Maniatis et al., Molecular Cloning: A Laboratory Manual (Cold Spring Harbor Press, 1982))

Once expressed, the proteins or glycoproteins having reduced O-linked glycosylation can be purified according to standard procedures of the art, including ammonium sulfate precipitation, affinity columns, column chromatography, gel electrophoresis and the like (See, generally, Scopes, R., Protein Purification (Springer-Verlag, N.Y., 1982)). Substantially pure glycoproteins of at least about 90 to 95% homogeneity are preferred, and 98 to 99% or more homogeneity most preferred, for pharmaceutical uses. Once purified, partially or to homogeneity as desired, the proteins can then be used therapeutically (including extracorporeally) or in developing and performing assay procedures, immunofluorescent stainings, and the like. (See, generally, Immunological Methods, Vols. I and II (Lefkovits and Pernis, eds., Academic Press, NY, 1979 and 1981).

Therefore, further provided are glycoprotein compositions comprising a predominant species of N-glycan structure and having reduced O-linked glycosylation compared to compositions of the glycoprotein which have been produced in host cells that have not been incubated in the presence of an inhibitor of Pmt-mediated O-linked glycosylation or an α-1,2-mannosidase capable of trimming more than one mannose residue from a glycan structure or both. In particular aspects, the glycoprotein composition comprises a glycoprotein having a predominant N-glycan structure selected from the group consisting of Man₅GlcNAc₂, GlcNAcMan₅GlcNAc₂, GalGlcNAcMan₅GlcNAc₂, NANAGalGlcNAcMan₅GlcNAc₂, GlcNAcMan₃GlcNAc₂, GlcNAc₍₁₋₄₎Man₃GlcNAc₂, Gal₍₁₋₄₎GlcNAc₍₁₋₄₎Man₃GlcNAc₂, and NANA₍₁₋₄₎Gal₍₁₋₄₎GlcNAc₍₁₋₄₎Man₃GlcNAc₂, wherein the subscript indicates the number of the particular sugar residues on the N-glycan structure. Examples of N-glycan structures include but are not limited to Man₅GlcNAc₂, GlcNAcMan₅GlcNAc₂, GlcNAcMan₃GlcNAc₂, GlcNAc₂Man₃GlcNAc₂, GlcNAc₃Man₃GlcNAc₂, GlcNAc₄Man₃GlcNAc₂, GalGlcNAc₂Man₃GlcNAc₂, Gal₂GlcNAc₂Man₃GlcNAc₂, Gal₂GlcNAc₃Man₃GlcNAc₂, Gal₂GlcNAc₄Man₃GlcNAc₂, Gal₃GlcNAc₃Man₃GlcNAc₂, Gal₃GlcNAc₄Man₃GlcNAc₂, Gal₄GlcNAc₄Man₃GlcNAc₂, NANAGal₂GlcNAc₂Man₃GlcNAc₂, NANA₂Gal₂GlcNAc₂Man₃GlcNAc₂, NANA₃Gal₃GlcNAc₃Man₃GlcNAc₂, and NANA₄Gal₄GlcNAc₄Man₃GlcNAc₂.

### III. Pharmaceutical Compositions

Proteins and glycoproteins having reduced O-linked glycosylation can be incorporated into pharmaceutical compositions comprising the glycoprotein as an active therapeutic agent and a variety of other pharmaceutically acceptable components (See, Remington's Pharmaceutical Science (15th ed., Mack Publishing Company, Easton, Pa., 1980). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation can also include other carriers, adjuvants, or nontoxic, nontherapeutic, nonimmunogenic stabilizers, and the like.

Pharmaceutical compositions for parenteral administration are sterile, substantially isotonic, pyrogen-free and prepared in accordance with GMP of the FDA or similar body. Glycoproteins can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Glycoproteins can be administered in the form of a depot injection or implant preparation which can be formulated in such a manner as to permit a sustained release of the active ingredient. Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above (See Langer, Science 249, 1527 (1990) and Hanes, Advanced Drug Delivery Reviews 28, 97-119 (1997).

Unless otherwise defined herein, scientific and technical terms and phrases used in connection with the present invention shall have the meanings that are commonly understood by those of ordinary skill in the art. Further, unless otherwise required by context, singular terms shall include the plural and plural terms shall include the singular. Generally, nomenclatures used in connection with, and techniques of biochemistry, enzymology, molecular and cellular biology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, for example, Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999).

The following examples are intended to promote a further understanding of the present invention.

### Example 1

To clone the α-1,2 mannosidase genes from various organisms, the catalytic domain of TrMds1 (amino acids 28-523 of GenBank Accession No. AAF34579) was blasted against the GenBank protein database to identify twelve α-1,2-mannosidases (MNS1) with amino acid similarity. See Table 1.

The catalytic domains for the twelve α-1,2-mannosidases identified as described above were fused at the N-terminus to α-MAT pre signal peptide (“αMATpreSS”; see SEQ ID Nos:1 and 2). The nucleotide sequences of certain α-1,2-mannosidases (codon-optimized for Pichia pastoris) were synthesized by GeneArt Inc., Regensburg, Germany. The nucleotide and amino acid sequences for the α-1,2-mannosidases from Coccidiodes immitis, Coccidiodes posadasi), Penicillium citrinum, Magnaporthe grisea, Aspergillus saitoi, Aspergillus oryzae, Chaetomiun globosum are shown in SEQ ID NOs: 3-16. The αMATss-MNS1 ORFs were subcloned into a vector designated pGLY2296 (comprising a Pichia pastoris AOX1 promoter, Pichia pastoris TRP integration site, and a URA5 selection marker).

Vector pGLY2269 is a double-crossover integration vector which contains a PpTRP1 ORF and, separately, nucleotides located immediately 3′ to the PpTRP1 ORF. The PpTRP1 ORF fragment (the 5′ arm) was generated by PCR using primers PpTRP1 A (SEQ ID NO: 17) and PpTRP1 B (SEQ ID NO: 18); the resulting DNA is shown (SEQ ID NO: 19 (PpTRP1 5′ arm)). The PpTRP1 3′ fragment (3′ arm) was generated by PCR using primers PpTRP1 C (SEQ ID NO: 20) and PpTRP1 D (SEQ ID NO: 21); the resulting DNA is shown (SEQ ID NO: 22 (PpTRP1 3′ arm)). The template was P. pastoris genomic DNA from wild-type strain NRRL-Y11430 (from Northern Regional Research Center, Peoria, Ill.). The PCR fragments were first cloned into pCR2.1 (Invitrogen, Carlsbad, Calif.) and sequenced. The PpTRP1 integration arms were then sub-cloned successively into pGLY566 which contains the URA5-LacZ blaster cassette (Nett and Gerngross, Yeast 20:1279 (2003), the PpALG3 transcriptional terminator sequence, and an expression cassette consisting of the PpAOX1 promoter (Cereghino and Cregg, FEMS Microbiol Rev. 24:45-66 (2000)) and ScCYC1 transcriptional terminator in pUC19 (New England Biolabs, Beverly, Mass.)), using enzymes Fse1 and SacI for the 5′ arm, and SpeI and SalI for the 3′ arm to generate pGLY2269. Situated just downstream of the PpTRP1 5′ arm is a DNA fragment encompassing the PpALG3 transcriptional terminator sequence, which was cloned by PCR using primers PpALG3TT-f (SEQ ID NO: 23) and PpALG3TT-rev (SEQ ID NO: 24) resulting in the DNA fragment shown (SEQ ID NO: 25; “ALGtt”). The PpALG3TT was sub-cloned using flanking Fse1 and Pme1 restriction sites. Situated between the PpTRP1 fragments in pGLY2269 are the URA5 marker (Nett and Gerngross, Yeast 20:1279 (2003), and an expression cassette consisting of the PpAOX1 promoter (Cereghino and Cregg, FEMS Microbiol Rev. 24:45-66 (2000)) and ScCYC1 transcriptional terminator separated by Not1 and Pac1 restriction sites. The PpAOX1 promoter sequence (SEQ ID NO: 26) and the ScCYC1 transcriptional terminator sequence (SEQ ID NO: 27) are shown. The αMATss-MNS1 ORFs were excised from vectors provided by GeneArt using Not1 and Pac1, and ligated into Not1 and Pac1 digested pGLY2269. Before transformation into yeast strains, the MNS1 plasmids were digested with SfiI which cuts at sites flanking the PpTRP1 sequences as described below in Example 2 (in section entitled “Co-transformation with αMATss-MNS1 ORFs”).

### Example 2

Pichia pastoris strains derived from wild-type strain NRRL-Y11430 (from Northern Regional Research Center, Peoria, Ill.) (as described below) were transformed with an expression vector encoding the heavy chain (Hc) and light chain (Lc) of the human α-Her2 antibody (Herceptin) and co-transformed with expression vectors encoding the α-1,2-mannosidases enzymes cloned as described in Example 1 produced a glycoprotein having reduced O-mannose chain length. GS115 is available from Invitrogen (Carlsbad, Calif.) and, with the exception of a HIS4 mutation to enable his4 selection, has an essentially wild type phenotype.

Anti-Her2 vector: Expression vector pGLY2988 contains expression cassettes under control of the methanol-inducible Pichia pastoris AOX1 promoter that encode the heavy (Hc) and light (Lc) chains of α-Her2. The Hc and Lc chains were generated using anti-Her2 antibody sequences obtained from GenBank. The GenBank accession number for the L chain is 1N8Z_A and the GenBank accession number for the H chain variable region plus CH1 domain is 1N8Z_B. The GenBank accession number for the H chain Fc region is BC092518. Both the Hc and Lc chain DNA sequences were codon optimized according to Pichia pastoris codon usage to enhance translation in Pichia pastoris. Qptimization of codons for use in Pichia sp. is well known in the art and has been described in, for example, Outchkourov et al., 2002, Protein Expr. Purif. 24:18-24; Sharp and Li, 1987, Nucleic Acids Res. 15:1281-95; Woo J H, Liu et al., 2002, Protein Expression and Purification 25:270-282, and, Nakamura, et al., 2000, Nucleic Acids Res. 28:292. Anti-Her2 Hc and Lc fused at the N-terminus to α-MATpre signal peptide (shown in SEQ ID Nos: 1 and 2) were synthesized by GeneArt Inc., Regensburg, Germany. Each was synthesized with unique 5′ EcoR1 and 3′ Fse1 sites. The codon-optimized nucleotide and amino acid sequences of the anti-Her2 Hc are shown in SEQ ID Nos: 28 and 29, respectively. The codon-optimized nucleotide and amino acid sequences of the anti-Her2 Lc are shown in SEQ ID Nos: 30 and 31, respectively. Both nucleic acid fragments encoding the Hc and Lc proteins fused to the α-MATpre signal peptide were separately subcloned using 5′ EcoR1 and 3′ Fse1 unique sites into an expression plasmid vector pGLY2198, which contains the Pichia pastoris TRP2 targeting nucleic acid and the Zeocin-resistance marker and generates expression cassettes under the control of the AOX1 promoter and Saccharomyces cerevisiae CYC terminator, to form plasmid vectors pGLY2987 and pGLY2338, respectively. The Lc expression cassette was then removed from plasmid vector pGLY2338 by digesting with BamHI and NotI and subcloned into plasmid vector pGLY2987 digested with BamH1 and Not1, thus generating the final expression plasmid vector pGLY2988.

The recipient strain for the α-1,2-mannosidase ORFs (produced as described in Example 1) was α-Her2 expression strain YGLY4282 which was constructed as follows: Five micrograms of pGLY2988 digested with restriction enzyme Spe1 which cuts in the TRP2 targeting region were used to transform the ura auxotrophic strain YGLY16-3. Strain YGLY16-3 (ura5Δ::ScSUC2 och1Δ::lacZ bmt2Δ::lacZ/KlMNN2-2/mnn4L1Δ::lacZ/MmSLC35A3 pno1Δmnn4Δ::lacZ), was constructed from wild-type strain NRRL-Y11430 (from Northern Regional Research Center, Peoria, Ill.) using methods described earlier (Nett and Gerngross, Yeast 20:1279 (2003); Choi et al., PNAS USA 100:5022 (2003); Hamilton et al., Science 301:1244 (2003)).

Transformation of YGLY16-3 was performed essentially as follows: YGLY16-3 was grown in YPD media (yeast extract (1%), peptone (2%), dextrose (2%)) overnight to an OD of between about 0.2 to 6. After incubation on ice for 30 minutes, cells were pelleted by centrifugation at 2500-3000 rpm for 5 minutes. Media was removed and the cells washed three times with ice cold sterile 1M sorbitol before resuspension in 0.5 ml ice cold sterile 1M sorbitol. Ten μL of linearized DNA (10 ug) and 100 μL cell suspension was combined in an electroporation cuvette and incubated for 5 minutes on ice. Electroporation was in a Bio-Rad GenePulser Xcell following the preset Pichia pastoris protocol (2 kV, 25 μF, 200Ω), immediately followed by the addition of 1 mL YPDS recovery media (YPD media plus 1 M sorbitol). The transformed cells were allowed to recover for four hours to overnight at room temperature (26° C.) before plating the cells on the selective media. Following selection on rich media containing zeocin, transformants were screened by small scale expression (Western analysis, described below) testing to detect α-Her2 expression. Strain YGLY4282 was selected based on high level α-Her2 expression.

Anti-Her2 protein expression was carried out by growing YGLY4282-derived strains in shake flasks or 96-deep well plates at 24° C. with buffered glycerol-complex medium (BMGY) consisting of 1% yeast extract, 2% peptone, 100 mM potassium phosphate buffer pH 6.0, 1.34% yeast nitrogen base, 4×10-5% biotin, and 1% glycerol. The induction medium for protein expression was buffered methanol-complex medium (BMMY) consisting of 1% methanol instead of glycerol in BMGY.

Co-transformation with αMATss-MNS1 ORFs described in Example 1: YGLY4282 was transformed with the αMATss-MNS1 ORFs as follows: The αMATss-MNS1 ORF plasmids described above in Example 1 were digested with SfiI which cuts at sites flanking the PpTRP1 sequences, thus freeing a DNA fragment containing the AOX1 promoter-αMATss-MNS1 ORF-transcriptional terminator plus URA5 marker flanked by PpTRP1 5′ and 3′ integration sequences. Transformations were as described above, except that the selection was on plates consisting of minimal media lacking uracil. Transformants harboring the properly integrated αMATss-MNS1 ORF were identified by colony PCR using the following three primer pairs: (i) primers corresponding to sequences 5′ and 3′ to the TRP1 sequences in pGLY2269 (SEQ ID Nos: 32 (TRP1 5′) and 33 (TRP1 3′), respectively), (ii) primers TRP1 5′ and to ALG3 sequences in pGLY2269 (SEQ ID NO: 34 (ALG3TT)), and (iii) primers TRP1 3′ to URA5 sequences in pGLY2269 (SEQ ID NO: 35 (URA5out)). PCR-grade genomic DNA was isolated by standard methods (Liang and Richardson, Biotechniques 13:730 (1992)). The PCR conditions were one cycle of 98° C. for 2 minutes; 30 cycles of 98° C. for 10 seconds, 50° C. for 30 seconds, and 72° C. for 1 minute; and finished by one cycle of 72° C. for 7 minutes. The PCR products were analyzed by agarose gel electrophoresis following standard methods.

Anti-Her2 Westerns: To test if the α-1,2-mannosidases cloned as described in Example 1 reduce O-mannose chain length on Pichia-produced recombinant protein, we tested if their co-expression altered the migration of the β-glycosylated α-Her2 heavy chain (Hc) as observed by SDS-PAGE as described in PCT Publication No. WO 2007/061631 A2. Transformants of YGLY4282 with the various αMATss-MNS1 ORFs were inoculated into 96-well deep well plates (Qiagen, Valencia, Calif.) containing 0.6 ml of BMGY media per well. After 24 hours growth with vigorous shaking, the 96-well plate was centrifuged at 2,000 rpm for five minutes to pellet cells. The media was removed and, following a wash step with 0.6 mL of BMMY media, the cells resuspended in 0.2 mL BMMY media. After an additional 24 hours growth with vigorous shaking, the plate was centrifuged at 2,000 rpm for five minutes to pellet cells, and the cleared supernatant subjected to Western blot analysis to detect α-Her2 expression. The Western blotting was performed as follows: seven μL of the supernatants were separated by reducing polyacrylamide gel electrophoresis (SDS-PAGE) according to Laemmli, U. K. (1970) Nature 227, 680-685 and then electroblotted onto nitrocellulose membranes (Schleicher & Schuell, now Whatman, Inc., Florham Park, N.J.). Anti-Her2 antibody chains were detected on the Western blots using a peroxidase-conjugated anti-human IgG (Hc+Lc) antibody (Calbiochem/EMD Biosciences, La Jolla, Calif.) and developed using the ImmunoPure Metal Enhanced DAB Substrate Kit (Pierce Biotechnology, Rockford, Ill.).

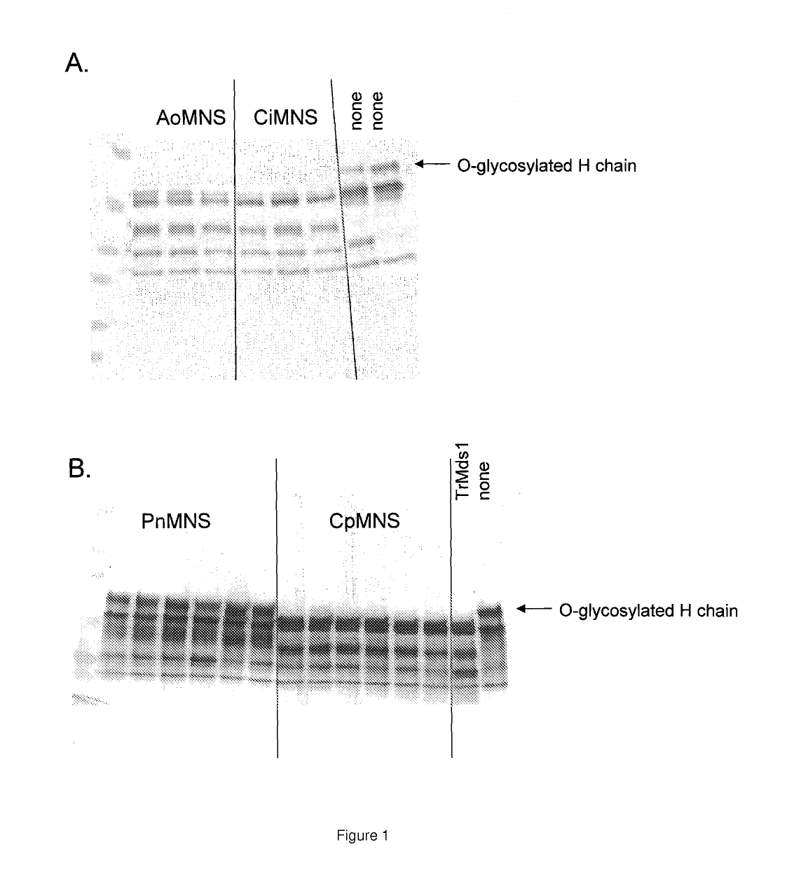

FIG. 1 shows the results of one such analysis in which transformants harboring C. immitis (CiMNS, FIG. 1, Panel A) or C. posadasii (CpMNS, FIG. 1, Panel B) MNS1, but not P. nodorum (PnMNS, FIG. 1, Panel B) MNS, expressed α-Her2 Hc that migrated faster than a non-transformed control. Hc migration in strains with the C. immitis and C. posadasii MNS was identical to that seen with T. reesei MDS1 (TrMds1, FIG. 1, Panel B). Similar results were observed for MNS from P. citrinum and M grisea (not shown). For A. saitoi (not shown), A. oryzae (AoMNS, FIG. 1, Panel A) and C. globosum (not shown) MNS1, the Hc migration was faster relative to the non-transformed control but not as fast as that observed with C. immitis, C. posadasii and T. reesei MNS1. Taken together, these results indicate that the MNS1 from C. immitis and C. posadasii are as active as T. reesei MDS1 in reducing O-mannose chain length on anti-Her2 Hc. Results are summarized in Table 1, third column.

To verify that the faster migration of α-Her2 Hc in the MNS transformants was due to reduced O-mannose chain length, we subjected antibody isolated from fermented MNS1 containing strains to O-mannose chain length analysis using high pH anion-exchange chromatography coupled with pulsed electrochemical detection-Dionex HPLC(HPAEC-PAD). YGLY16-3 based strains expressing α-Her2 plus T. reesei, C. immitis, C. posadasii, P. citrinum, M grisea, A. saitoi, A. oryzae, and C. globosum MNS1 were fermented in 0.5 L vessels as follows: Bioreactor Screenings (SIXFORS) were done in 0.5 L vessels (Sixfors multi-fermentation system, ATR Biotech, Laurel, Md.) under the following conditions: pH at 6.5, 24° C., 0.3 SLPM, and an initial stirrer speed of 550 rpm with an initial working volume of 350 mL (330 mL BMGY medium and 20 mL inoculum). IRIS multi-fermenter software (ATR Biotech, Laurel, Md.) was used to linearly increase the stirrer speed from 550 rpm to 1200 rpm over 10 hours, one hour after inoculation. Seed cultures (200 mL of BMGY in a 1 L baffled flask) were inoculated directly from agar plates. The seed flasks were incubated for 72 hours at 24° C. to reach optical densities (OD₆₀₀) between 95 and 100. The fermenters were inoculated with 200 mL stationary phase flask cultures that were concentrated to 20 mL by centrifugation. The batch phase ended on completion of the initial charge glycerol (18-24 h) fermentation and were followed by a second batch phase that was initiated by the addition of 17 mL of glycerol feed solution (50% [w/w] glycerol, 5 mg/L Biotin, 12.5 mL/L PTM1 salts (65 g/L FeSO₄.7H₂O, 20 g/L ZnCl₂, 9 g/L H₂SO₄, 6 g/L CuSO₄.5H₂O, 5 g/L H₂SO₄, 3 g/L MnSO₄.7H₂O, 500 mg/L CoCl₂.6H₂O, 200 mg/L NaMoO₄.2H₂O, 200 mg/L biotin, 80 mg/L NaI, 20 mg/L H₃BO₄)). Upon completion of the second batch phase, as signaled by a spike in dissolved oxygen, the induction phase was initiated by feeding a methanol feed solution (100% MeOH 5 mg/L biotin, 12.5 mL/L PTM1) at 0.6 g/h for 32-40 hours. The cultivation is harvested by centrifugation.

Anti-Her2 was purified from cleared supernatant by protein A chromatography (Li et al. Nat. Biotechnol. 24(2):210-5 (2006)), and O-glycan determination was performed using Dionex-HPLC(HPAEC-PAD) as follows: O-glycans were released and separated from protein by alkaline elimination (beta-elimination) (Harvey, Mass Spectrometry Reviews 18: 349-451 (1999), Stadheim et al., Nat. Protoc. 3:1026-31 (2006)). This process also reduces the newly formed reducing terminus of the released O-glycan (either oligomannose or mannose) to mannitol. The mannitol group thus serves as a unique indicator of each O-glycan. 0.5 nmole or more of protein, contained within a volume of 100 μL PBS buffer, was required for beta elimination. The sample was treated with 25 μL alkaline borohydride reagent and incubated at 50° C. for 16 hours. About 20 uL arabitol internal standard was added, followed by 10 μL glacial acetic acid. The sample was then centrifuged through a Millipore filter containing both SEPABEADS and AG 50W-X8 resin and washed with water. The samples, including wash, were transferred to plastic autosampler vials and evaporated to dryness in a centrifugal evaporator. 150 μL1% AcOH/MeOH was added to the samples and the samples evaporated to dryness in a centrifugal evaporator. This last step was repeated five more times. 200 μL of water was added and 100 μL of the sample was analyzed by high pH anion-exchange chromatography coupled with pulsed electrochemical detection-Dionex HPLC(HPAEC-PAD) according to the manufacturer (Dionex, Sunnyvale, Calif.).

The results of this analysis are shown in the right-most column of Table 1. The results showed that T reesei, C. immitis, and C. posadasi MNS1 significantly increased the percentage of O-Man1 relative to no MNS1 control. MNS1 from P. citrinum, M grisea, A. saitoi, A. oryzae and C. globosum also reduced O-mannose chain length, but to a lesser extent.

TABLE 1
α-1,2-mannosidase screening for O-mannose reduction

| | Accession | O-mannosidase | O-Man1 |
|---|---|---|---|
| mannosidase | number | activity^{a} | (%)^{b} |
| none | — | − | 8 |
| Trichoderma reesei | AAF34579 | ++ | 95 |
| Coccidiodes immitis | EAS32290 | ++ | 96 |
| Coccidiodes posadasii | ABA54911 | ++ | 97 |
| Penicillium citrinum | BAA08275 | ++ | 83 |
| Magnaporthe grisea | XP_368250 | ++ | 87 |
| Aspergillus saitoi | BAA08634 | + | 86 |
| Aspergillus oryzae | BAE57653 | + | 89 |
| Chaetomiun globosum | XP_001227052 | + | 89 |
| Dictyostelium discoideum | XP_629442 | − | Not |
| | | | done (ND) |
| Coprinopsis cinerea | XP_001840635 | − | ND |
| Gibberella zeae | XP_384082 | − | ND |
| Phaeosphaeria nodorum | EAT80794 | − | ND |
| Neurospora crassa | XP_960332 | − | ND |

^{a}determined by faster migration of O-glycosylated α-Her2 Hc on Western blots (FIG. 1).
^{b}determined by HPEAC-PAD analysis of O-glycans released from α-Her2.

### Example 3

To test if C. immitis MNS reduced O-mannose chain length for a broad range of Pichia-expressed antibodies, we co-expressed the AOX1p-αMATpreSS-CiMNS vector in strains expressing anti-Respiratory Syncytial virus (α-RSV, i.e. Synagis), anti-tumor necrosis factor alpha (α-TNFα, i.e., Humira), and anti-Vascular endothelial growth factor (α-VEGF, i.e., Avastin) along with our α-Her2 control. For each antibody, the Lc variable region plus kappa chain constant region, and the Hc variable region plus IgG constant regions were fused to the αMATpre signal sequence, and gene synthesis was performed by GeneArt AG as described above for our α-Her2 antibody. αMATpreSS-Lc and αMATpreSS-Hc DNA (codon optimized for expression in P. pastoris) and amino sequences for each antibody are shown in SEQ ID NOs: 36-47. Subcloning of the Lc and Hc genes into vector pGLY2198 was as described above for α-Her2. To facilitate this analysis, strain YGLY17108 was generated that harbored the AOX1p-αMATpreSS-CiMNS construct, and then transformed with vectors encoding the Lc plus Hc for each antibody. YGLY17108 was made by transforming the AOX1p-αMATpreSS-CiMNS vector described above into strain YGLY5858 which contains enzymes required for galactose synthesis and transfer to complex glycans on heterologousproteins (Bobrowicz et al., Glycobiology 14: 757 (2004); Hamilton et al., Science 313:1441 (2006); Li et al., Nat. Biotechnol. 24:210-5 (2006)). YGLY5858 was derived as described for YAS309 (Li et al., Nat. Biotechnol. 24:210-5 (2006)) except that it is a uracil auxotroph. As a control, YGLY5858 was also transformed with an AOX1p-αMATpreSS-TrMds1 vector, then the resulting parental strain received the α-Her2 expression vector pGLY2988 as described above. Strains were fermented in 0.5 L vessels, and protein purified by protein A chromatography and subjected to O-glycan analysis by HPAEC-PAD as described above. Results (shown in Table 2 below) indicated that CiMNS effectively reduced O-mannose chain length on all antibodies tested to 100% O-Man1.

TABLE 2
CiMNS reduces O-mannose to O-Man1 on multiple antibodies.

| | | O-Man1^{a} |
|---|---|---|
| Antibody | O-mannosidase | (%) |
| α-Her2 | TrMds1 (control) | 100 |
| α-Her2 | CiMNS | 100 |
| α-RSV | CiMNS | 100 |
| α-TNF α | CiMNS | 100 |
| α-VEGF | CiMNS | 100 |

^{a}determined by HPEAC-PAD analysis of O-glycans released from antibodies listed. Values represent those determined for multiple transformants for each group.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

### SEQUENCES

SEQ ID NO 1: Amino acid sequence of the aMATpreSS
MRFPSIFTAVLFAASSALA
SEQ ID NO: 2: Nucleic acid sequence of the
aMATpreSS
ATGAGATTCCCATCCATCTTCACTGCTGTTTTGTTCGCTGCTTCTTCTG
CTTTGGCT
SEQ ID NO: 3: Nucleic acid sequence (codon
optimized for expression in P. pastoris) for
C. immitis α-1,2-mannosidase
TTGCCAATGATTGACAAGCACTTGCCATCCTCTATTGGTCAATCCTCCT
ACAAGACTTCCAGAGAAAGAGCTGAGGCTGTTAAGGACGCTTTTAGATT
CGCTTGGAACGGTTACTTGGAACACGCTTTCCCAAACGATGAATTGCAC
CCAGTTTCCAACACTCCAGGAAACTCTAGAAACGGTTGGGGAGCTTCTG
CTGTTGATGCTTTGTCCACTGCTATCATCATGGACATGCCAGACGTTGT
TGAGAAGATTTTGGACCACATCTCCAACATTGACTACTCCCAGACTGAC
ACTATGTGTTCCTTGTTCGAGACTACTATCAGATACTTGGGTGGAATGA
TCTCTGCTTACGACTTGTTGAAGGGTCCAGGTTCTCACTTGGTTTCTGA
CCCAGCTAAGGTTGACGTTTTGTTGGCTCAGTCCTTGAAGTTGGCTGAC
GTTTTGAAGTTCGCTTTCGACACTAAGACTGGTATTCCAGCTAACGAGT
TGAACATCACTGACAAGTCTACTGACGGTTCCACTACAAACGGATTGGC
TACTACTGGTACTTTGGTTTTGGAGTGGACTAGATTGTCCGACATTACT
GGTGATCCAGAGTACGGTAGATTGGCTCAAAAGGGTGAGTCCTACTTGT
TGAACCCACAACCATCTTCCTCTGAACCATTCCCAGGATTGGTTGGTAG
AACTATCGACATCGAGACTGGTTTGTTCAGAGATGACTACGTTTCTTGG
GGTGGTGGTTCTGACTCTTTCTACGAGTACTTGATCAAGATGTACGTTT
ACGACAAGGACAGATTCGGTAAGTACAAAGAGAGATGGGTTACAGCTGC
TGAGTCCACTATCAAGCACTTGAAGTCCTCTCCATCCACTAGAAAGGAC
TTGACTTTCGTTGCTACTTACTCCGGTGGTAGATTGGGATTGAACTCCG
GTCACTTGACTTGTTTCGACGGTGGTAACTTCTTGTTGGGTGGTCAAAT
CTTGGACAGAGATGACTTCACTAAGTTCGGATTGGAGTTGGTTGAAGGA
TGTTACGCTACTTACGCTGCTACTGCTACTAAGATTGGTCCAGAGGGTT
TTGGATGGGACGATACTAAGGTTCCAGAAGCTCAGGCTGAGTTCTACAA
AGAGGCTGGTTTCTACATCACTACTTCCTACTACAACTTGAGACCAGAG
GTTATCGAGTCCATCTACTACGCTTACAGAATGACTAAGGACCCAAAGT
ACCAAGAATGGGCTTGGGATGCTTTTGTTGCTATCAACGCTACTACTAG
AACTTCCACTGGTTTCACTGCTATCGGTGACGTTAACACTCCAGACGGT
GGTAGAAAGTACGACAACCAAGAGTCCTTCTTGTTCGCTGAGGTTATGA
AGTACTCCTACTTGATTCACTCTCCAGAAGCTGATTGGCAAGTTGCTGG
TCCAGGTGGTACTAATGCTTACGTTTTCAACACTGAGGCTCACCCAGTT
AAGGTTTTCTCCAGAGGTTGT
SEQ ID NO: 4: Amino acid sequence of C. immitis
α-1,2-mannosidase
LPMIDKHLPSSIGQSSYKTSRERAEAVKDAFRFAWNGYLEHAFPNDELH
PVSNTPGNSRNGWGASAVDALSTAIIMDMPDVVEKILDHISNIDYSQTD
TMCSLFETTIRYLGGMISAYDLLKGPGSHLVSDPAKVDVLLASQLKLAD
VLKFAFDTKTGIPANELNITDKSTDGSTTNGLATTGTLVLEWTRLSDIT
GDPEYGRLAQKGESYLLNPQPSSSEPFPGLVGRTIDIETGLFRDDYVSW
GGGSDSFYEYLIKMYVYDKDRFGKYKERWVTAAESTIKHLKSSPSTRKD
LTFVATYSGGRLGLNSGHLTCFDGGNFLLGGQILDRDDFTKFGLELVEG
CYATYAATATKIGPEGFGWDDTKVPEAQAEFYKEAGFYITTSYYNLRPE
VIESIYYAYRMTKDPKYQEWAWDAFVAINATTRTSTGFTAIGDVNTPDG
GRKYDNQESFLFAEVMKYSYLIHSPEADWQVAGPGGTNAYVFNTEAHPV
KVFSRGC
SEQ ID NO: 5: Nucleic acid sequence (codon
optimized for expression in P. pastoris) for C.
posadasii α-1,2-mannosidase
TTGCCAATGATTGACAAGCACTTGCCATCCTCTATTGGTCAATCCTCCT
ACAAGACTTCCAGAGAAAGAGCTGAGGCTGTTAAGGACGCTTTTAGATT
CGCTTGGAACGGTTACTTGGAACACGCTTTCCCAAACGATGAATTGCAC
CCAGTTTCCAACACTCCAGGAAACTCTAGAAACGGTTGGGGAGCTTCTG
CTGTTGATGCTTTGTCCACTGCTATCATCATGGACATGCCAGACGTTGT
TGAGAAGATTTTGGACCACATCTCCAACATTGACTACTCCCAGACTGAC
ACTATGTGTTCCTTGTTCGAGACTACTATCAGATACTTGGGTGGAATGA
TCTCTGCTTACGACTTGTTGAAGGGTCCAGGTTCTCACTTGGTTTCTGA
CCCAGCTAAGGTTGACGTTTTGTTGGCTCAGTCCTTGAAGTTGGCTGAC
GTTTTGAAGTTCGCTTTCGACACTAAGACTGGTATTCCAGCTAACGAGT
TGAACATCACTGACAAGTCTACTGACGGTTCCACTACAAACGGATTGGC
TACTACTGGTACTTTGGTTTTGGAGTGGACTAGATTGTCCGACATTACT
GGTGATCCAGAGTACGGTAGATTGGCTCAAAAGGGTGAGTCCTACTTGT
TGAACCCACAACCATCTTCCTCTGAACCATTCCCAGGATTGGTTGGTAG
AACTATCGACATCGAGACTGGTTTGTTCAGAGATGACTACGTTTCTTGG
GGTGGTGGTTCTGACTCTTTCTACGAGTACTTGATCAAGATGTACGTTT
ACGACAAGGACAGATTCGGTAAGTACAAAGAGAGATGGGTTACAGCTGC
TGAGTCCACTATCAAGCACTTGAAGTCCTCTCCATCCACTAGAAAGGAC
TTGACTTTCGTTGCTACTTACTCCGGTGGTAGATTGGGATTGAACTCCG
GTCACTTGACTTGTTTCGACGGTGGTAACTTCTTGTTGGGTGGTCAAAT
CTTGGACAGAGATGACTTCACTAAGTTCGGATTGGAGTTGGTTGAAGGA
TGTTACGCTACTTACGCTGCTACTGCTACTAAGATTGGTCCAGAGGGTT
TTGGATGGGACGATACTAAGGTTCCAGAAGCTCAGGCTGAGTTCTACAA
AGAGGCTGGTTTCTACATCACTACTTCCTACTACAACTTGAGACCAGAG
GTTATCGAGTCCATCTACTACGCTTACAGAATGACTAAGGACCCAAAGT
ACCAAGAATGGGCTTGGGATGCTTTTGTTGCTATCAACGCTACTACTAG
AACTTCCACTGGTTTCACTGCTATCGGTGACGTTAACACTCCAGACGGT
GGTAGAAAGTACGACAACCAAGAGTCCTTCTTGTTCGCTGAGGTTATGA
AGTACTCCTACTTGATTCACTCTCCAGAAGCTGATTGGCAAGTTGCTGG
TCCAGGTGGTACTAATGCTTACGTTTTCAACACTGAGGCTCACCCAGTT
AAGGTTTTCTCCAGAGGTTGT
SEQ ID NO: 6: Amino acid sequence for C.
posadasii α-1,2-mannosidase
LPMIDKDLPSSISQSSDKTSQERAEAVKDAFRFAWEGYLEHAFPNDELH
PVSNTPGNSRNGWGASAVDALSTAIIMDMPDVVEKILKHISNIDYSQTD
TMCSLFETTIRYLGGMISAYDLLKGPGSHLVSDPAKVDVLLAQSLKLAD
VLKFAFDTKTGIPANELNITDKSTDGSTTNGLATTGTLVLEWTRLSDIT
GDPEYGRLAQKGESYLLNPQPSSSEPFPGLVGRTIDIETGLFRDDYVSW
GGGSDSFYEYLIKMYVYDKGRFGKYKDRWVTAAESTIEHLKSSPSTRKD
LTFVATYSGGRLGLNSGHLTCFDGGNFLLGGQILNRDDFTKFGLELVEG
CYATYAATATKIGPEGFGWDATKVPEAQAEFYKEAGFYITTSYYNLRPE
VIESIYYAYRMTKDPKYQEWAWDAFVAINATTRTSTGFTAIGDVNTPDG
GRKYDNQESFLFAEVMKYSYLIHSPEADWQVAGPGGTNAYVFNTEAHPV
KVFSRGC
SEQ ID NO: 7: Nucleic acid sequence (codon
optimized for expression in P. pastoris) for
Penicillium citrinum α-1,2-mannosidase
CACCCATACGGTGAAACTGAGGCTGTTTTGAGATCCGAACCAAAGTCCA
ACCAAGCTAAGGCTGACGCTGTTAAAGAGGCTTTTCAGCACGCTTGGAA
CGGTTACATGAAGTACGCTTTCCCACACGATGAATTGACTCCAGTTTCC
AACGGTCACGCTGATTCTAGAAATGGATGGGGAGCTTCTGCTGTTGATG
CTTTGTCCACTGCTGTTATCATGGGAAAGGCTGATGTTGTTAACGCTAT
CTTGGAGCACGTTGCTGACATTGACTTCTCCAAGACTTCCGACACTGTT
TCCTTGTTCGAGACTACTATCAGATACTTGGCTGGAATGTTGTCTGGTT
ACGACTTGTTGCAAGGTCCAGCTAAGAACTTGGTTGACAACCAGGACTT
GATCGATGGTTTGTTGGACCAGTCCAGAAACTTGGCTGACGTTTTGAAG
TTCGCTTTCGACACTCCATCTGGTGTTCCATACAACAACATCAACATCA
CTTCCCACGGTAATGATGGTGCTACTACAAACGGATTGGCTGTTACTGG
TACTTTGGTTTTGGAGTGGACTAGATTGTCTGACTTGACTGGTGACGAA
GAGTACGCTAAGTTGTCCCAAAAGGCTGAGTCCTACTTGTTGAAGCCAC
AACCATCTTCCTCTGAACCATTCCCAGGATTGGTTGGTTCCTCCATCAA
CATTAACGACGGTCAATTCGCTGACTCTAGAGTTTCTTGGAACGGTGGT
GACGACTCTTTCTACGAGTACTTGATCAAGATGTACGTTTACGACCCAA
AGAGATTCGAGACTTACAAGGACAGATGGGTTTTGGCTGCTGAGTCCAC
TATTAAGCACTTGAAGTCCCACCCAAAGTCTAGACCAGACTTGACTTTC
TTGTCCTCCTACTCCAACAGAAACTACGACTTGTCCTCCCAACACTTGA
CTTGTTTCGACGGTGGTTCCTTTTTGTTGGGTGGTACTGTTTTGGACAG
ACAGGACTTCATTGACTTCGGATTGGAGTTGGTTGATGGTTGTGAGGCT
ACTTACAACTCCACTTTGACTAAGATTGGTCCAGATTCTTGGGGATGGG
ACCCAAAGAAGGTTCCATCCGACCAGAAAGAGTTCTACGAGAAGGCTGG
TTTCTACATTTCTTCCGGTTCCTACGTTTTGAGACCAGAGGTTATCGAG
TCCTTCTACTACGCTCACAGAGTTACTGGTAAAGAAATCTACAGAGACT
GGGTTTGGAACGCTTTCGTTGCTATCAACTCCACTTGTAGAACTGACTC
TGGTTTCGCTGCTGTTTCCGACGTTAACAAGGCTAACGGTGGTTCTAAG
TACGACAACCAAGAGTCCTTCTTGTTCGCTGAGGTTATGAAGTACTCCT
ACTTGGCTCACTCTGAAGATGCTGCTTGGCAAGTTCAAAAGGGTGGAAA
GAACACTTTCGCTTTACAACACTGAGGCTCACCCAATTTCCGTTGCTAG
AAAC
SEQ ID NO: 8: Amino acid sequence for Penicillium
citrinum α-1,2-mannosidase
HPYGETEAVLRSEPKSNQAKADAVKEAFQHAWNGYMKYAFPHDELTPVS
NGHADSRNGWGASAVDALSTAVIMGKADVVNAILEHVADIDFSKTSDTV
SLFETTIRYLAGMLSGYDLLQGPAKNLVDNQDLIKGLLDQSRNLADVLK
FAFDTPSGVPYNNINITSHGNDGATTNGLAVTGTLVLEWTRLSDLTGDE
EYAKLSQKAESYLLKPQPSSSEPFPGLVGSSININDGQFADSRVSWNGG
DDSFYEYLIKMYVYDPKRFETYKDRWVLAAESTIKHLKSHPKSRPDLTF
LSSYSNRNYDLSSQHLTCFDGGSFLLGGTVLDRQDFIDFGLELVDGCEA
TYNSTLTKIGPDSWGWDPKKVPDDQKEFYEKAGFYISSGSYVLRPEVIE
SFYYAHRVTGKEIYRDWVWNAFVAINSTCRTDSGFAAVSDVNKANGGSK
YDNQESFLFAEVMKYSYLAHSEDAAWQVQKGGKNTFVYNTEAHPISVAR
N
SEQ ID NO: 9: Nucleic acid sequence (codon
optimized for expression in P. pastoris) for
Magnaporthe grisea α-1,2-mannosidase
TCCCCATTGACTAAGAGAGCTATCCAGTACTCTCCAAACCCAGAAAAGG
CTGAGGCTGTTAAGGCTGCTTTTAGAAGATCCTGGGACGGTTACATGCA
ACATGCTTTCCCACACGACACTTTGAGACCAGTTTCTCAAGGATGGGCT
GATGATAGAAATGGATGGGGAGCTTCTGCTGTTGATGCTTTCGGTACTG
CTATCGTTATGGACGAAGAGGCTATCGTTCAGCAAATCTTGGACTTCGT
TCCAACTATCGACTTCGACAAGACTTCTACTGAGGTTTCCTTGTTCGAG
ACTACTATCAGATACTTGGGAGGATTGGTTTCTGCTTACGACTTGTTGA
CTGAGCCAAACGGTAAACACGCTACTGGTAACGAGACTTTGGTTAAGGC
TGTTTTGTCCCAGGCTGAGAGATTGGCTACAAACTTGAAGTTCGCTTTC
GACACTCCATCTGGTATTCCAGACAACTCCGTTTACTTCAACCCATCCA
GAAGAGGAGGTTCCCAAACTAACGGTATCGCTACTATCGGTACTTTGCA
GTTGGAGTGGACTAGATTGGGAGACTTGACTGGTAACCCAGTTTTCACT
GAGTTGGCTTTGAAGGCTAACTCCTACTTGTTGGACCCAAAGCCAGCTT
TGGGTGAACCATTCCCTGGTTTGTTGGGAACTAACGTTAACATCACTAC
TGGTTTGTTCACTGACGGTAATGGTGGATGGGGTGGTGGTACTGACTCT
TTCTACGAGTACTTGATCAAGATGTACTTGTACGACCCAGAGAGATTCG
GTCAATACAGAGACAGATGGGTTTTGGCTGCTGACTCCTCCATTAAGTA
CATGGCTTCCCACCCATCTACTAGACCAGACTTGACTTTCTTGGCTGAG
TGGAGAGACACTTCCTTGAGATACATCTCCCAGCACTTGGCTTGTTTTG
ACGGTGGTAACTTCATCTTGGGAGGATTGACTTTGCAGAACCAGAAGTA
CGTTGACTTCGGATTGGCTTTGACTAACGCTTGTCACGAGACTTACATC
CAGACTGTTACTGGTATTGGTCCAGAGGTTTTCAGATGGCAAGATGCTT
CTGCTGCTAACTCTTCCACTAATCCACCTGCTCCAGCTGGATTGCAAGA
GTTCTACAAGAGAGCTGGTTTCTGGATTCCACCATCCAACGATATTGGT
GGTGCTGCTATCTACCAATTGAGACCAGAGGTTATCGAGTCCTTCTACT
ACGCTTACAGAGCTACTGGTGACACTAAGTACCAAGACTGGGCTTGGGA
GGCTTTCTTGTCCATCAACAGAACTTGTGCTGCTGGTACTGCTTACTCT
TCCATCTCCGACGTTAATGCTCCAGGTGGTGGTTCTTCTTTGGACTTCC
AAGAGTCCTTTTGGTTCGCTGAGACTATGAAGTACTCCTACTTGATTCA
CGCTGAAGATGCTCCATGGCAAGTTAAGGCTGACAGAACTAACGGATGG
GTTTTCAACACTGAGGCTCACCCAATTAAGGTTCACGGTAGACCAGGT
SEQ ID NO: 10: Amino acid sequence for
Magnaporthe grisea α-1,2-mannosidase
SPLTKRAIQYSPNPEKAEAVKAAFRRSWDGYMQHAFPHDTLRPVSQGWA
DDRNGWGASAVDAFGTAIVMDEEAIVQQILDFVPTIDFDKTSTEVSLFE
TTIRYLGGLVSAYDLLTEPNGKHATGNETLVKAVLSQAERLATNLKFAF
DTPSGIPDNSVYFNPSRRGGSQTNGIATIGTLQLEWTRLGDLTGNPVFT
ELALKANSYLLDPKPALGEPFPGLLGTNVNITTGLFTDGNGGWGGGTDS
FYEYLIKMYLDPERFGQYRDRWVLAADSSIKYMASHPSTRPDLTFLAEW
RDTSLRYISQHLACFDGGNFILGGLTLQNQKYVDFGLALTNACHETYIQ
TVTGIGPEVFRWQDASAANSSTNPPAPGLQEFYKRAGFWIPPSNDIGGA
AIYQLRPEVIESFYYAYRATGDTKYQDWAWEAFLSINRTCAAGTAYSSI
SDVNAPGGGSSLDFQESFWFAETMKYSYLIHAEDAPWQVKADRTNGWVF
NTEAHPIKVHGRPG
SEQ ID NO: 11: Nucleic acid sequence (codon
optimized for expression in P. pastoris) for A.
saitoi α-1,2-mannosidase
TACCCACATTTCGGTTCCTCTCAGCCAGTTTTGCACTCTTCTTCCGACA
CTACTCAATCTAGAGCTGACGCTATCAAGGCTGCTTTTTCTCATGCTTG
GGACGGTTACTTGCAATACGCTTTCCCACACGATGAATTGCACCCAGTT
TCCAACGGTTACGGTGACTCTAGAAATGGATGGGGAGCTTCTGCTGTTG
ATGCTTTGTCCACTGCTGTTATCATGAGAAACGCTACTATCGTTAACCA
AATCTTGGACCATGTTGGAAAGATCGACTACTCCAAGACTAACACTACT
GTTTCCTTGTTCGAGACTACTATCAGATACTTGGGTGGAATGTTGTCTG
GTTACGACTTGTTGAAGGGTCCAGTTTCCGATTTGGTTCAGAACTCCTC
CAAGATCGACGTTTTGTTGACTCAGTCCAAGAACTTGGCTGACGTTTTG
AAGTTCGCTTTCGACACTCCATCTGGTGTTCCATACAACAACTTGAACA
TCACTTCCGGTGGTAATGATGGTGCTAAGACTAACGGATTGGCTGTTAC
TGGTACTTTGGCTTTGGAGTGGACTAGATTGTCTGACTTGACTGGTGAC
ACTACTTACGCTGACTTGTCCCAAAAGGCTGAGTCCTACTTGTTGAACC
CACAACCAAAGTCTGCTGAACCATTCCCAGGATTGGTTGGTTCCAACAT
CAACATCTCCAACGGTCAATTCACTGACGCTCAGGTTTCATGGAATGGT
GGTGACGACTCTTACTACGAGTACTTGATCAAGATGTACGTTTACGACC
CAAAGAGATTCGGTTTGTACAAGGACAGATGGGTTGCTGCTGCTCAATC
TACTATGCAGCACTTGGCTTCTCATCCATCTTCTAGACCAGACTTGACT
TTCTTGGCTTCCTACAACAACGGTACTTTGGGTTTGTCCTCCCAGCATT
TGACTTGTTTCGACGGTGGTTCCTTTTTGTTGGGTGGTACTGTTTTGAA
CAGAACTGACTTCATCAACTTCGGATTGGACTTGGTTTCTGGTTGTCAC
GACACTTACAACTCCACTTTGACTGGTATTGGTCCAGAATCTTTCTCTT
GGGACACTTCCGACATTCCATCTTCCCAACAGTCCTTGTACGAAAAGGC
TGGTTTCTACATCACTTCTGGTGCTTACATCTTGAGACCAGAGGTTATC
GAGTCCTTCTACTACGCTTGGAGAGTTACTGGTCAAGAGACTTACAGAG
ACTGGATTTGGTCTGCTTTCTCCGCTGTTAACGACTACTGTAGAACTTC
TTCCGGTTTCTCCGGATTGACTGACGTTAATGCTGCTAATGGTGGTTCC
AGATACGACAACCAAGAGTCCTTCTTGTTCGCTGAGGTTATGAAGTACT
CCTACATGGCTTTCGCTGAAGATGCTGCTTGGCAAGTTCAACCAGGTTC
CGGTAACCAGTTCGTTTTCAACACTGAGGCTCACCCAGTTAGAGTTTCT
TCCACT
SEQ ID NO: 12: Amino acid sequence for A. saitoi
α-1,2-mannosidase
YPHFGSSQPVLHSSSDTTQSRADAIKAAFSHAWDGYLQYAFPHDELHPV
SNGYGDSRNGWGASAVDALSTAVIMRNATIVNQILDHVGKIDYSKTNTT
VSLFETTIRYLGGMLSGYDLLKGPVSDLVQNSSKIDVLLTQSKNLADVL
KFAFDTPSGVPYNNLNITSGGNDGAKTNGLAVTGTLALEWTRLSDLTGD
TTYADLSQKAESYLLNPQPKSAEPFPGLVGSNINISNGQFTDAQVSWNG
GDDSYYEYLIKMYVYDPKRFGLYKDRWVAAAQSTMQHLASHPSSRPDLT
FLASYNNGTLGLSSQHLTCFDGGSFLLGGTVLNRTDFINFGLDLVSGCH
DTYNSTLTGIGPESFSWDTSDIPSSQQSLYEKAGFYITSGAYILRPEVI
ESFYYAWRVTGQETYRDWIWSAFSAVNDYCRTSSGFSGLTDVNAANGGS
RYDNQESFLFAEVMKYSYMAFAEDAAWQVQPGSGNQFVFNTEAHPVRVS
ST
SEQ ID NO: 13: Nucleic acid sequence (codon
optimized for expression in P. pastoris) for A.
oryzae α-1,2-mannosidase
TACCCACAATTCAAGTTCGAGCAGAGAGTTGCTAGATCCAACTCTTCTG
AGTCCAGAGCTAACGCTGTTAAAGAGGCTTTTGTTCACGCTTGGGACGG
TTACATGCAATACGCTTACCCACACGATGAATTGCACCCAATCTCCAAC
GGTGTTGGTGATTCTAGAAACGGATGGGGAGCTTCTGCTGTTGATGCTT
TGTCCACTGCTGTTATCATGGGTAACGAGACTATCGTTAACCAAATCTT
GGACCACATCGCTACTATTGACTACTCCAAGACTGACGACCAAGTTTCC
TTGTTCGAGACTACTATCAGATACTTGGGTGGAATGTTGTCTGGTTACG
ACTTGTTGAAGGGTCCAGCTTCCAACTTGGTTAAGGACCAGGCTAAGGT
TAAGACTTTGTTGGACCAGTCCCAAAACTTGGCTGACGTTTTGAAGTTC
GCTTTCGACACTCCATCTGGTATCCCATACAACAACATCAACATCACTT
CCCACGGTAATGATGGTGCTACTACAAACGGATTGGCTGTTACTGGTAC
TTTGGTTTTGGAGTGGACTAGATTGTCTGACTTGACTGGTGACACTGAG
TACGCTCAATTGTCCCAAAAGGCTGAGGACTACTTGTTGAACCCATCTC
CAAAGTCTGCTGAACCATTCGAAGGATTGGTTGGTTCCCACATCAACAT
TTCCAACGGTGCTTTCGCTGATGGTCAAGTTTCTTGGAACGGTGGTGAC
GACTCTTTCTACGAGTACTTGATCAAGATGTACGTTTACGACCCAAAGA
GATTCTCTACTTACGGTGACAGATGGGTTAAGGCTGCTGAGTCCTCCAT
TAAGCACTTGGCTTCTCACCCAGAAAAGAGACCAGACTTGACTTTCTTG
GCTTCCTACAACGATGGTCAGTACGGTTTGTCCTCCCAACACTTGACTT
GTTTCGACGGTGGTTCCTTTTTGTTGGGTGGTACTGTTTTGGACAGAGA
TGACTTCATCCAGTTCGGATTGGATTTGGTTAAGGGTTGTCACGAGACT
TACAACCAGACTTTGACTGGTATTGGTCCAGAATCTTTCGGATGGGACC
CAAAGAATGTTCCATCCGACCAGAAAGAGTTGTACGAGAGAGCTGGTTT
CTACATTTCCTCCGGTGCTTACATTTTGAGACCAGAGGTTATCGAGTCC
TTCTACTACGCTTGGAGAATCACTGGACAAGAAATCTACAGAGAATGGG
TTTGGAACGCTTTCGTTAACATCAACAAGTACTGTAGAACTGACTCTGG
TTTCGCTGGATTGACTAACGTTAACGCTGCTAACGGTGGTGGAAGATAC
GACAACCAAGAGTCCTTCTTGTTCGCTGAGGTTTTGAAATACGTTTACT
TGACTTTCGCTCCAGACAACGAGTGGCAAGTTCAAAGAGGAAAGGGTAA
CAAGTTCGTTTACAACACTGAGGCTCACCCAGTTAGAGTTGCTGCT
SEQ ID NO: 14: Amino acid sequence for A. oryzae
α-1,2-mannosidase
YPQFKFEQRVARSNSSESRANAVKEAFVHAWDGYMQYAYPHDELHPISN
GVGDSRNGWGASAVDALSTAVIMGNETIVNQILDHIATIDYSKTDDQVS
LFETTIRYLGGMLSGYDLLKGPASNLVKDQAKVKTLLDQSQNLADVLKF
AFDTPSGIPYNNINITSHGNDGATTNGLAVTGTLVLEWTRLSDLTGDTE
YAQLSQKAEDYLLNPSPKSAEPFEGLVGSHINISNGAFADGQVSWNGGD
DSFYEYLIKMYVYDPKRFSTYGDRWVKAAESSIKHLASHPEKRPDLTFL
ASYNDGQYGLSSQHLTCFDGGSFLLGGTVLDRDDFIQFGLDLVKGCHET
YNQTLTGIGPESFGWDPKNVPSDQKELYERAGFYISSGAYILRPEVIES
FYYAWRITGQEIYREWVWNAFVNINKYCRTDSGFAGLTNVNAANGGGRY
DNQESFLFAEVLKYVYLTFAPDNEWQVQRGKGNKFVYNTEAHPVRVAA
SEQ ID NO: 15: Nucleic acid sequence (codon
optimized for expression in P. pastoris) for C.
globosum α-1,2-mannosidase
TTGCCAAGATTGGAAGATGGTGGTAGACCAGGTGCTGGTAGAGAAGATT
CTTTCTGGTCTAAGGTTCCATTGCACTACCCACCAGACTCTATTAGACC
ATTGCCAACTGGATTGCCAGTTCAATACCCACCAATTCAAACTACTACT
TTCCCAGCTGAAGAAGATCAAGCTGCTAGAGGTAGAAGAGTTCAAAGAC
AAAGGCTGTTAGAGATGTTTTCGCTAAGTGTTGGGCTTCTTACAGAAAG
TTGGCTTGGGGTGCTGATGAATTGACTCCAGTTTCTGGTGGTAGAAAGG
ACCCATTTGGTGGATGGTCAGCTACTTTGGTTGACTCTTTGGACACTTT
GTGGATTATGGACATGAAAACTGAATTTGACGAAGCTGTTGCTGCTGCT
GACGGTATTAACTTCACTCACACTGCTGTTGACAGAGTTAACGTTTTCG
AAACTAACATTAGATACCTTGGTGGTTTCTTGGCTGCTTTCGACTTGTC
TGGTGACAAGAGATTGTTGGGAAAGGCTACAGAGGTCGGAGACATGTTG
TACAAGGCTTTCGACACTCCATCTCACATGCCAATTACTAGATGGGACA
TGAGAGCTGCTATTAAGGGTGCTAAACAAGTTGCTTCTGAAGGATTGAT
TGCTGAAGTCGGAACTTTGTCTATGGAGTTCACTAGATTGTCTATGTTG
ACTGGTGACCCAAAGTGGTTCGACTTGGCTCAAAGAATTACTGACGACA
TGGCTGCTCAACAAGACTCTACTGCTGTTCCTGGTTTGTGGCCATTGAA
GGTTAACGGTCAAAAGAGAATTTTCAACTCTGGTCCAGACTTTACTTTG
GGTGCTATGGCTGACTCTGCTTACGAATACTTGCCAAAGATGTCTGCTA
TGATGGGTGGTCAATTGCCAATGTACCAAACTATGTACGAAAAGGCTAT
GGACGCTGCTACTAAGCACAACTTGTTCAGACCAATGACTCCAACTAAC
GAAGATATTTTGATTGCTGGTGACGTTCACGCTAGAGAAAACGGAATGG
AATTTGAAGCTAGAGGTCAACACTTGACTTGTTTCCTTGGTGGATTGAT
GGCTTTGGGTGGTAGATTGTACGGTAGAGAAAAGGACGTTATTGCTGGT
AGAAAGTTGACTGACGGTTGTGTTTGGACTTATAAAGCATTTCCACAAG
GTATTATGCCAGAATCTTTCGTTACTGTTTCTTGTCCAGCTGGTGATGC
TTGTGAATGGGACGAGGGCGTCTGGAAGAAAGAAGTTTTGAGAAAGGCT
AACAAGGATACTGACGGTCCAGAATCTAACGCTGAAGCTGAAGTTATCA
TTAAGAAGCAAAGATTGCCACAAGGTTTCACAGCTGTGTTCGACAGAGC
TTACTTGTTGAGACCAGAAGCTATTGAATCTGTTTTCGTTTTGTACAGA
GTTACTGGAAAGGTTGAATTGTTGGAAGCTGCTTGGGATATGTTCACTG
CTATTGACAAGGCTACATCTACAGACTTGGCTAATTCTGCTATCAGGGA
CGTCACATCCACTGATAAGCCAGAGGCTAAAGACTCTATGGAATCTTTC
TGGATGGCTGAAACTTTGAAGTACTTCTACTTGATTTTCTCTGACCCAG
AATTGATTAACTTGAACGAATACGTTTTGAACACTGAAGCTCACCCATT
CAGAAGATTGTTGCCATCTCCA
SEQ ID NO: 16: Amino acid sequence for C.
globosum α-1,2-mannosidase
LPRLEDGGRPGAGREDSFWSKVPLHYPPDSIRPLPTGLPVQYPPIQTTT
FPAEEDQAARGRRVQRQKAVRDVFAKCWASYRKLAWGADELTPVSGGRK
DPFGGWSATLVDSLDTLWIMDMKTEFDEAVAAADGINFTHTAVDRVNVF
ETNIRYLGGFLAAFDLSGDKRLLGKATEVGDMLYKAFDTPSHMPITRWD
MRAAIKGAKQVASEGLIAEVGTLSMEFTRLSMLTGDPKWFDLAQRITDD
MAAQQDSTAVPGLWPLKVNGQKRIFNSGPDFTLGAMADSAYEYLPKMSA
MMGGQLPMYQTMYEKAMDAATKHNLFRPMTPTNEDILIAGDVHARENGM
EFEARGQHLTCFLGGLMALGGRLYGREKDVIAGRKLTDGCVWTYKAFPQ
GIMPESFVTVSCPAGDACEWDEGVWKKEVLRKANKDTDGPESNAEAEVI
IKKQRLPQGFTAVFDRAYLLRPEAIESVFVLYRVTGKVELLEAAWDMFT
AIDKATSTDLANSAIRDVTSTDKPEAKDSMESFWMAETLKYFYLIFSDP
ELINLNEYVLNTEAHPFRRLLPSP
SEQ ID NO: 17: Primer PpTRP1 A
GAGCTCGGCCTTGGAGGCCGCGGAAACGGCAGTAAACAATGGAGCTTCA
SEQ ID NO: 18: Primer PpTRP1 B
GGCCGGCCTTATGTAGCGTTTCTGATAAATTGTGTGAT
SEQ ID NO: 19: PpTRP1 5′ arm
GAGCTCGGCCTTGGAGGCCGCGGAAACGGCAGTAAACAATGGAGCTTCA
TTAGTGGGTGTTATTATGGTCCCTGGCCGGGAACGAACGGTGAAACAAG
AGGTTGCGAGGGAAATTTCGCAGATGGTGCGGGAAAAGAGAATTTCAAA
GGGCTCAAAATACTTGGATTCCAGACAACTGAGGAAAGAGTGGGACGAC
TGTCCTCTGGAAGACTGGTTTGAGTACAACGTGAAAGAAATAAACAGCA
GTGGTCCATTTTTAGTTGGAGTTTTTCGTAATCAAAGTATAGATGAAAT
CCAGCAAGCTATCCACACTCATGGTTTGGATTTCGTCCAACTACATGGG
TCTGAGGATTTTGATTCGTATATACGCAATATCCCAGTTCCTGTGATTA
CCAGATACACAGATAATGCCGTCGATGGTCTTACCGGAGAAGACCTCGC
TATAAATAGGGCCCTGGTGCTACTGGACAGCGAGCAAGGAGGTGAAGGA
AAAACCATCGATTGGGCTCGTGCACAAAAATTTGGAGAACGTAGAGGAA
AATATTTACTAGCCGGAGGTTTGACACCTGATAATGTTGCTCATGCTCG
ATCTCATACTGGCTGTATTGGTGTTGACGTCTCTGGTGGGGTAGAAACA
AATGCCTCAAAAGATATGGACAAGATCACACAATTTATCAGAAACGCTA
CATAAGGCCGGCC
SEQ ID NO: 20: Primer PpTRP1 C
ACTAGTATTTAAATAAGTCAATTAAATACACGCT
SEQ ID NO: 21: Primer PpTRP1 D
GTCGACGGCCAAGACGGCCAAGTCGTATCTCCTTTCCACGACATCCCA
SEQ ID NO: 22: PpTRP1 3′ Arm
ACTAGTATTTAAATAAGTCAATTAAATACACGCTTGAAAGGACATTACA
TAGCTTTCGATTTAAGCAGAACCAGAAATGTAGAACCACTTGTCAATAG
ATTGGTCAATCTTAGCAGGAGCGGCTGGGCTAGCAGTTGGAACAGCAGA
GGTTGCTGAAGGTGAGAAGGATGGAGTGGATTGCAAAGTGGTGTTGGTT
AAGTCAATCTCACCAGGGCTGGTTTTGCCAAAAATCAACTTCTCCCAGG
CTTCACGGCATTCTTGAATGACCTCTTCTGCATACTTCTTGTTCTTGCA
TTCACCAGAGAAAGCAAACTGGTTCTCAGGTTTTCCATCAGGGATCTTG
TAAATTCTGAACCATTCGTTGGTAGCTCTCAACAAGCCCGGCATGTGCT
TTTCAACATCCTCGATGTCATTGAGCTTAGGAGCCAATGGGTCGTTGAT
GTCGATGACGATGACCTTCCAGTCAGTCTCTCCCTCATCCAACAAAGCC
ATAACACCGAGGACCTTGACTTGCTTGACCTGTCCAGTGTAACCTACGG
CTTCACCAATTTCGCAAACGTCCAATGGATCATTGTCACCCTTGGCCTT
GGTCTCTGGATGAGTGACGTTAGGGTCTTCCCATGTCTGAGGGAAGGCA
CCGTAGTTGTGAATGTATCCGTGGTGAGGGAAACAGTTACGAACGAAAC
GAAGTTTTCCCTTCTTTGTGTCCTGAAGAATTGGGTTCAGTTTCTCCTC
CTTGGAAATCTCCAACTTGGCGTTGGTCCAACGGGGGACTTCAACAACC
ATGTTGAGAACCTTCTTGGATTCGTCAGCATAAAGTGGGATGTCGTGGA
AAGGAGATACGACTTGGCCGTCTTGGCCGTCGAC
SEQ ID NO: 23: Primer PpALG3TT-f
GGCCGGCCATTTACAATTAGTAATATTAAGGT
SEQ ID NO: 24: Primer PpALG3TT-rev
GTTTAAACCTACTAAGCGACGAAAACGGGA
SEQ ID NO: 25: ALGtt
GGCCGGCCATTTACAATTAGTAATATTAAGGTGGTAAAAACATTCGTAG
AATTGAAATGAATTAATATAGTATGACAATGGTTCATGTCTATAAATCT
CCGGCTTCGGTACCTTCTCCCCAATTGAATACATTGTCAAAATGAATGG
TTGAACTATTAGGTTCGCCAGTTTCGTTATTAAGAAAACTGTTAAAATC
AAATTCCATATCATCGGTTCCAGTGGGAGGACCAGTTCCATCGCCAAAA
TCCTGTAAGAATCCATTGTCAGAACCTGTAAAGTCAGTTTGAGATGAAA
TTTTTCCGGTCTTTGTTGACTTGGAAGCTTCGTTAAGGTTAGGTGAAAC
AGTTTGATCAACCAGCGGCTCCCGTTTTCGTCGCTTAGTAGGTTTAAAC
SEQ ID NO: 26: PpAOX1 promoter sequence
CTCGAGAGATCTAACATCCAAAGACGAAAGGTTGAATGAAACCTTTTTG
CCATCCGACATCCACAGGTCCATTCTCACACATAAGTGCCAAACGCAAC
AGGAGGGGATACACTAGCAGCAGACCGTTGCAAACGCAGGACCTCCACT
CCTCTTCTCCTCAACACCCACTTTTGCCATCGAAAAACCAGCCCAGTTA
TTGGGCTTGATTGGAGCTCGCTCATTCCAATTCCTTCTATTAGGCTACT
AACACCATGACTTTATTAGCCTGTCTATCCTGGCCCCCCTGGCGAGGTT
CATGTTTGTTTATTTCCGAATGCAACAAGCTCCGCATTACACCCGAACA
TCACTCCAGATGAGGGCTTTCTGAGTGTGGGGTCAAATAGTTTCATGTT
CCCCAAATGGCCCAAAACTGACAGTTTAAACGCTGTCTTGGAACCTAAT
ATGACAAAAGCGTGATCTCATCCAAGATGAACTAAGTTTGGTTCGTTGA
AATGCTAACGGCCAGTTGGTCAAAAAGAAACTTCCAAAAGTCGGCATAC
CGTTTGTCTTGTTTGGTATTGATTGACGAATGCTCAAAAATAATCTCAT
TAATGCTTAGCGCAGTCTCTCTATCGCTTCTGAACCCCGGTGCACCTGT
GCCGAAACGCAAATGGGGAAACACCCGCTTTTTGGATGATTATGCATTG
TCTCCACATTGTATGCTTCCAAGATTCTGGTGGGAATACTGCTGATAGC
CTAACGTTCATGATCAAAATTTAACTGTTCTAACCCCTACTTGACAGCA
ATATATAAACAGAAGGAAGCTGCCCTGTCTTAAACCTTTTTTTTTATCA
TCATTATTAGCTTACTTTCATAATTGCGACTGGTTCCAATTGACAAGCT
TTTGATTTTAACGACTTTTAACGACAACTTGAGAAGATCAAAAAACAAC
TAATTATTCGAAACGGCGGCCGC
SEQ ID NO: 27: ScCYC1 terminator sequence
TTAATTAAACAGGCCCCTTTTCCTTTGTCGATATCATGTAATTAGTTAT
GTCACGCTTACATTCACGCCCTCCTCCCACATCCGCTCTAACCGAAAAG
GAAGGAGTTAGACAACCTGAAGTCTAGGTCCCTATTTATTTTTTTTAAT
AGTTATGTTAGTATTAAGAACGTTATTTATATTTCAAATTTTTCTTTTT
TTTCTGTACAAACGCGTGTACGCATGTAACATTATACTGAAAACCTTGC
TTGAGAAGGTTTTGGGACGCTCGAAGGCTTTAATTTGCAAGCTGCCGGC
TCTTAA
SEQ ID NO: 28: Anti-Her2 Heavy chain (DNA)
GAGGTTCAGTTGGTTGAATCTGGAGGAGGATTGGTTCAACCTGGTGGTT
CTTTGAGATTGTCCTGTGCTGCTTCCGGTTTCAACATCAAGGACACTTA
CATCCACTGGGTTAGACAAGCTCCAGGAAAGGGATTGGAGTGGGTTGCT
AGAATCTACCCAACTAACGGTTACACAAGATACGCTGACTCCGTTAAGG
GAAGATTCACTATCTCTGCTGACACTTCCAAGAACACTGCTTACTTGCA
GATGAACTCCTTGAGAGCTGAGGATACTGCTGTTTACTACTGTTCCAGA
TGGGGTGGTGATGGTTTCTACGCTATGGACTACTGGGGTCAAGGAACTT
TGGTTACTGTTTCCTCCGCTTCTACTAAGGGACCATCTGTTTTCCCATT
GGCTCCATCTTCTAAGTCTACTTCCGGTGGTACTGCTGCTTTGGGATGT
TTGGTTAAAGACTACTTCCCAGAGCCAGTTACTGTTTCTTGGAACTCCG
GTGCTTTGACTTCTGGTGTTCACACTTTCCCAGCTGTTTTGCAATCTTC
CGGTTTGTACTCTTTGTCCTCCGTTGTTACTGTTCCATCCTCTTCCTTG
GGTACTCAGACTTACATCTGTAACGTTAACCACAAGCCATCCAACACTA
AGGTTGACAAGAAGGTTGAGCCAAAGTCCTGTGACAAGACTCATACTTG
TCCACCATGTCCAGCTCCAGAATTGTTGGGTGGTCCTTCCGTTTTTTTG
TTCCCACCAAAGCCAAAGGACACTTTGATGATCTCCAGAACTCCAGAGG
TTACATGTGTTGTTGTTGACGTTTCTCACGAGGACCCAGAGGTTAAGTT
CAACTGGTACGTTGACGGTGTTGAAGTTCACAACGCTAAGACTAAGCCA
AGAGAGGAGCAGTACAACTCCACTTACAGAGTTGTTTCCGTTTTGACTG
TTTTGCACCAGGATTGGTTGAACGGAAAGGAGTACAAGTGTAAGGTTTC
CAACAAGGCTTTGCCAGCTCCAATCGAAAAGACTATCTCCAAGGCTAAG
GGTCAACCAAGAGAGCCACAGGTTTACACTTTGCCACCATCCAGAGATG
AGTTGACTAAGAACCAGGTTTCCTTGACTTGTTTGGTTAAGGGATTCTA
CCCATCCGACATTGCTGTTGAATGGGAGTCTAACGGTCAACCAGAGAAC
AACTACAAGACTACTCCACCTGTTTTGGACTCTGACGGTTCCTTTTTCT
TGTACTCCAAGTTGACTGTTGACAAGTCCAGATGGCAACAGGGTAACGT
TTTCTCCTGTTCCGTTATGCATGAGGCTTTGCACAACCACTACACTCAA
AAGTCCTTGTCTTTGTCCCCTGGTAAGTAA
SEQ ID NO: 29: Anti-Her2 Heavy chain (protein)
EVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSR
WGGDGFYAMDYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK
SEQ ID NO: 30: Anti-Her2 light chain (DNA)
GACATCCAAATGACTCAATCCCCATCTTCTTTGTCTGCTTCCGTTGGTG
ACAGAGTTACTATCACTTGTAGAGCTTCCCAGGACGTTAATACTGCTGT
TGCTTGGTATCAACAGAAGCCAGGAAAGGCTCCAAAGTTGTTGATCTAC
TCCGCTTCCTTCTTGTACTCTGGTGTTCCATCCAGATTCTCTGGTTCCA
GATCCGGTACTGACTTCACTTTGACTATCTCCTCCTTGCAACCAGAAGA
TTTCGCTACTTACTACTGTCAGCAGCACTACACTACTCCACCAACTTTC
GGACAGGGTACTAAGGTTGAGATCAAGAGAACTGTTGCTGCTCCATCCG
TTTTCATTTTCCCACCATCCGACGAACAGTTGAAGTCTGGTACAGCTTC
CGTTGTTTGTTTGTTGAACAACTTCTACCCAAGAGAGGCTAAGGTTCAG
TGGAAGGTTGACAACGCTTTGCAATCCGGTAACTCCCAAGAATCCGTTA
CTGAGCAAGACTCTAAGGACTCCACTTACTCCTTGTCCTCCACTTTGAC
TTTGTCCAAGGCTGATTACGAGAAGCACAAGGTTTACGCTTGTGAGGTT
ACACATCAGGGTTTGTCCTCCCCAGTTACTAAGTCCTTCAACAGAGGAG
AGTGTTAA
SEQ ID NO: 31: Anti-Her2 light chain (protein)
DIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTF
GQGTKVEIKRTVAAPSVFIFPPSDEQLKSGTASVVCLNNFYPREAKVQW
KVDNALQSGNSQESVTEQDSKDSTYSLSSTLTLSKADYEKHKVYACEVT
HQGLSSPVTKSFNRGEC
SEQ ID NO: 32: Primer TRP1 5′
GGA AGC TCG AGT TTA AGC GGT GCT G
SEQ ID NO: 33: Primer TRP1 3′
GAT TGC CTC CTG ATT ACG AAG AAG CCG
SEQ ID NO: 34: Primer ALG3TT
CTA CTA AGC GAC GAA AAC GGG AGC CG
SEQ ID NO: 35: Primer URA5out
GGG AGA GTT GAA GGT TGT ATT ATT GCC
SEQ ID NO: 36: α-RSV Lc (DNA)
ATGAGATTCCCATCCATCTTCACTGCTGTTTTGTTCGCTGCTTCTTCTG
CTTTGGCTGACATTCAGATGACACAGTCCCCATCTACTTTGTCTGCTTC
CGTTGGTGACAGAGTTACTATCACTTGTAAGTGTCAGTTGTCCGTTGGT
TACATGCACTGGTATCAGCAAAAGCCAGGAAAGGCTCCAAAGTTGTTGA
TCTACGACACTTCCAAGTTGGCTTCCGGTGTTCCATCTAGATTCTCTGG
TTCCGGTTCTGGTACTGAGTTCACTTTGACTATCTCTTCCTTGCAACCA
GATGACTTCGCTACTTACTACTGTTTCCAGGGTTCTGGTTACCCATTCA
CTTTCGGTGGTGGTACTAAGTTGGAGATCAAGAGAACTGTTGCTGCTCC
ATCCGTTTTCATTTTCCCACCATCCGACGAACAATTGAAGTCCGGTACC
GCTTCCGTTGTTTGTTTGTTGAACAACTTCTACCCACGTGAGGCTAAGG
TTCAGTGGAAGGTTGACAACGCTTTGCAATCCGGTAACTCCCAAGAATC
CGTTACTGAGCAGGATTCTAAGGATTCCACTTACTCATTGTCCTCCACT
TTGACTTTGTCCAAGGCTGATTACGAGAAGCACAAGGTTTACGCTTGCG
AGGTTACACATCAGGGTTTGTCCTCCCCAGTTACTAAGTCCTTCAACAG
AGGAGAGTGTTAATAG
SEQ ID NO: 37: α-RSV light chain (protein)
M R F P S I F T A V L F A A S S A L A D I Q M T Q
S P S T L S A S V G D R V T I T C K C Q L S V G Y
M H W Y Q Q K P G K A P K L L I Y D T S K L A S G
V P S R F S G S G S G T E F T L T I S S L Q P D D
F A T Y Y C F Q G S G Y P F T F G G G T K L E I K
R T V A A P S V F I F P P S D E Q L K S G T A S V
V C L L N N F Y P R E A K V Q W K V D N A L Q S G
N S Q E S V T E Q D S K D S T Y S L S S T L T L S
K A D Y E K H K V Y A C E V T H Q G L S S P V T K
S F N R G E C
SEQ ID NO: 38: α-RSV heavy chain (DNA)
ATGAGATTCCCATCCATCTTCACTGCTGTTTTGTTCGCTGCTTCTTCTG
CTTTGGCTCAGGTTACATTGAGAGAATCCGGTCCAGCTTTGGTTAAGCC
AACTCAGACTTTGACTTTGACTTGTACTTTCTCCGGTTTCTCCTTGTCT
ACTTCCGGAATGTCTGTTGGATGGATCAGACAACCACCTGGAAAGGCTT
TGGAATGGCTTGCTGACATTTGGTGGGATGACAAGAAGGACTACAACCC
ATCCTTGAAGTCCAGATTGACTATCTCCAAGGACACTTCCAAGAATCAA
GTTGTTTTGAAGGTTACAAACATGGACCCAGCTGACACTGCTACTTACT
ACTGTGCTAGATCCATGATCACTAACTGGTACTTCGATGTTTGGGGTGC
TGGTACTACTGTTACTGTCTCGAGTGCTTCTACTAAGGGACCATCCGTT
TTTCCATTGGCTCCATCCTCTAAGTCTACTTCCGGTGGAACCGCTGCTT
TGGGATGTTTGGTTAAAGACTACTTCCCAGAGCCAGTTACTGTTTCTTG
GAACTCCGGTGCTTTGACTTCTGGTGTTCACACTTTCCCAGCTGTTTTG
CAATCTTCCGGTTTGTACTCTTTGTCCTCCGTTGTTACTGTTCCATCCT
CTTCCTTGGGTACTCAGACTTACATCTGTAACGTTAACCACAAGCCATC
CAACACTAAGGTTGACAAGAGAGTTGAGCCAAAGTCCTGTGACAAGACA
CATACTTGTCCACCATGTCCAGCTCCAGAATTGTTGGGTGGTCCATCCG
TTTTCTTGTTCCCACCAAAGCCAAAGGACACTTTGATGATCTCCAGAAC
TCCAGAGGTTACATGTGTTGTTGTTGACGTTTCTCACGAGGACCCAGAG
GTTAAGTTCAACTGGTACGTTGACGGTGTTGAAGTTCACAACGCTAAGA
CTAAGCCAAGAGAAGAGCAGTACAACTCCACTTACAGAGTTGTTTCCGT
TTTGACTGTTTTGCACCAGGACTGGTTGAACGGTAAAGAATACAAGTGT
AAGGTTTCCAACAAGGCTTTGCCAGCTCCAATCGAAAAGACTATCTCCA
AGGCTAAGGGTCAACCAAGAGAGCCACAGGTTTACACTTTGCCACCATC
CAGAGAAGAGATGACTAAGAACCAGGTTTCCTTGACTTGTTTGGTTAAA
GGATTCTACCCATCCGACATTGCTGTTGAGTGGGAATCTAACGGTCAAC
CAGAGAACAACTACAAGACTACTCCACCAGTTTTGGATTCTGATGGTTC
CTTCTTCTTGTACTCCAAGTTGACTGTTGACAAGTCCAGATGGCAACAG
GGTAACGTTTTCTCCTGTTCCGTATGCATGAGGCTTTGCACAACCACTA
CACTCAAAAGTCCTTGTCTTTGTCCCCTGGTTAATGA
SEQ ID NO: 39: α-RSV Heavy chain (protein)
M R F P S I F T A V L F A A S S A L A Q V T L R E
S G P A L V K P T Q T L T L T C T F S G F S L S T
S G M S V W I R Q P P G K A L E W L A D I W W D D
K K A D Y N P S L K S R L T I S K D T S K N Q V V
L K V T N M D P A D T A T Y Y C A R S M I T N W Y
F D V W G A G T T V T V S S A S T K G P S V F P L
A P S S K S T S G G T A A L G C L V K D Y F P E P
V T V S W N S G A L T S G V H T F P A V L Q S S G
L Y S L S S V V T V P S S S L G T Q T Y I C N V N
H K P S N T K V D K R V E P K S C D K T H T C P P
C P A P E L L G G P S V F L F P P K P K D T L M I
S R T P E V T C V V V D V S H E D P E V K F N W Y
V D G V E V H N A K T K P R E E Q Y N S T Y R V V
S V L T V L H Q D W L N G K E Y K C K V S N K A L
P A P I E K T I S K A K G Q P R E P Q V Y T L P P
S R E E M T K N Q V S L T C L V K G F Y P S D I A
V E W E S N G Q P E N N Y K T T P P V L D S D G S
F F L Y S K L T V D K S R W Q Q G N V F S C S V M
H E A L H N H Y T Q K S L S L S P G
SEQ ID NO: 40: α-TNFα light chain (DNA)
ATGAGATTCCCATCCATCTTCACTGCTGTTTTGTTCGCTGCTTCTTCTG
CTTTGGCTGACATCCAAATGACTCAATCCCCATCTTCTTTGTCTGCTTC
CGTTGGTGACAGAGTTACTATCACTTGTAGAGCTTCCCAGGGTATCAGA
AACTACTTGGCTTGGTATCAACAGAAGCCAGGAAAGGCTCCAAAGTTGT
TGATCTACGCTGCTTCCACTTTGCAATCTGGTGTTCCATCTAGATTCTC
TGGTTCCGGTTCTGGTACTGACTTCACTTTGACTATCTCTTCCTTGCAA
CCAGAGGACGTTGCTACTTACTACTGTCAGAGATACAACAGAGCACCAT
ACACTTTCGGACAGGGTACTAAGGTTGAGATCAAGAGAACTGTTGCTGC
TCCATCCGTTTTCATTTTCCCACCATCCGACGAACAATTGAAGTCCGGT
ACCGCTTCCGTTGTTTGTTTGTTGAACAACTTCTACCCACGTGAGGCTA
AGGTTCAGTGGAAGGTTGACAACGCTTTGCAATCCGGTAACTCCCAAGA
ATCCGTTACTGAGCAGGATTCTAAGGATTCCACTTACTCATTGTCCTCC
ACTTTGACTTTGTCCAAGGCTGATTACGAGAAGCACAAGGTTTACGCTT
GCGAGGTTACACATCAGGGTTTGTCCTCCCCAGTTACTAAGTCCTTCAA
CAGAGGAGAGTGTTAATAG
SEQ ID NO: 41: α-TNFα light chain (protein)
MRFPSIFTAVLFAASSALADIQMTQSPSSLSASVGDRVTITCRASQGIR
NYLAWYQQKPGKAPKLLIYAASTLQSGVPSRFSGSGSGTDFTLTISSLQ
PEDVATYYCQRYNRAPYTFGQGTKVEIKRTVAAPSVFIFPPSDEQLKSG
TASVVCLLNNFYPREAKVQWKVDNALQSGNSQESVTEQDSKDSTYSLSS
TLTLSKADYEKHKVYACEVTHQGLSSPVTKSFNRGEC
SEQ ID NO: 42: α-TNFα heavy chain (DNA)
ATGAGATTCCCATCCATCTTCACTGCTGTTTTGTTCGCTGCTTCTTCTG
CTTTGGCTGAGGTTCAATTGGTTGAATCCGGTGGTGGATTGGTTCAACC
TGGTAGATCCTTGAGATTGTCCTGTGCTGCTTCCGGTTTTACTTTCGAC
GACTACGCTATGCATTGGGTTAGACAGGCTCCAGGTAAAGGATTGGAAT
GGGTTTCCGCTATTACTTGGAACTCCGGTCACATTGATTACGCTGACTC
CGTTGAGGGAAGATTCACTATCTCCAGAGACAACGCTAAGAACTCCTTG
TACTTGCAGATGAACTCCTTGAGAGCTGAGGATACTGCTGTTTACTACT
GTGCTAAGGTTTCCTACTTGTCCACTGCTTCTTCCTTGGATTACTGGGG
ACAGGGAACTTTGGTTACTGTCTCGAGTGCTTCTACTAAGGGACCATCC
GTTTTTCCATTGGCTCCATCCTCTAAGTCTACTTCCGGTGGAACCGCTG
CTTTGGGATGTTTGGTTAAAGACTACTTCCCAGAGCCAGTTACTGTTTC
TTGGAACTCCGGTGCTTTGACTTCTGGTGTTCACACTTTCCCAGCTGTT
TTGCAATCTTCCGGTTTGTACTCTTTGTCCTCCGTTGTTACTGTTCCAT
CCTCTTCCTTGGGTACTCAGACTTACATCTGTAACGTTAACCACAAGCC
ATCCAACACTAAGGTTGACAAGAAGGTTGAGCCAAAGTCCTGTGACAAG
ACTCATACTTGTCCACCATGTCCAGCTCCAGAATTGTTGGGTGGTCCTT
CCGTTTTTTTGTTCCCACCAAAGCCAAAGGACACTTTGATGATCTCCAG
AACTCCAGAGGTTACATGTGTTGTTGTTGACGTTTCTCACGAGGACCCA
GAGGTTAAGTTCAACTGGTACGTTGACGGTGTTGAAGTTCACAACGCTA
AGACTAAGCCAAGAGAGGAGCAGTACAACTCCACTTACAGAGTTGTTTC
CGTTTTGACTGTTTTGCACCAGGATTGGTTGAACGGAAAGGAGTACAAG
TGTAAGGTTTCCAACAAGGCTTTGCCAGCTCCAATCGAAAAGACTATCT
CCAAGGCTAAGGGTCAACCAAGAGAGCCACAGGTTTACACTTTGCCACC
ATCCAGAGATGAGTTAACTAAGAACCAGGTTTCCTTGACTTGTTTGGTT
AAGGGATTCTACCCATCCGACATTGCTGTTGAATGGGAGTCTAACGGTC
AACCAGAGAACAACTACAAGACTACTCCACCTGTTTTGGACTCTGACGG
TTCCTTTTTCTTGTACTCCAAGTTGACTGTTGACAAGTCCAGATGGCAA
CAGGGTAACGTTTTCTCCTGTTCCGTTATGCATGAGGCTTTGCACAACC
ACTACACTCAAAAGTCCTTGTCTTTGTCCCCTGGTTAATGA
SEQ ID NO: 43: α-TNFα heavy chain (protein)
MRFPSIFTAVLFAASSALAEVQLVESGGGLVQPGRSLRLSCAASGFTFD
DYAMHWVRQAPGKGLEWVSAITWNSGHIDYADSVEGRFTISRDNAKNSL
YLQMNSLRAEDTAVYYCAKVSYLSTASSLDYWGQGTLVTVSSASTKGPS
VFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDK
THTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYK
CKVSNKALPAPIEKTISKAKGQPREPQVYTLPPSRDELTKNQVLSLTCL
VKGFYPSDIAVEWESNGQPENNYKTTPPVLDSDGSFFLYSKLTVDKSRW
QQGNVFSCSVMHEALHNHYTQKSLSLSLPG
SEQ ID NO: 44: α-VEGF light chain (DNA)
ATGAGATTCCCATCCATCTTCACTGCTGTTTTGTTCGCTGCTTCTTCTG
CTTTGGCTGACATCCAAATGACACAATCCCCATCTTCCTTGTCTGCTTC
CGTTGGTGACAGAGTTACTATCACTTGTTCCGCTTCCCAAGACATCTCC
AACTACTTGAACTGGTATCAGCAGAAGCCAGGTAAAGCTCCAAAGGTTT
TGATCTACTTCACTTCTTCCTTGCACTCTGGTGTTCCATCTAGATTCTC
TGGTTCCGGTTCTGGTACTGACTTCACTTTGACTATCTCTTCCTTGCAA
CCAGAGGACTTCGCTACTTACTACTGTCAGCAGTACTCTACTGTTCCAT
GGACTTTCGGACAGGGTACTAAGGTTGAGATCAAGAGAACTGTTGCTGC
TCCATCCGTTTTCATTTTCCCACCATCCGACGAACAATTGAAGTCCGGT
ACCGCTTCCGTTGTTTGTTTGTTGAACAACTTCTACCCACGTGAGGCTA
AGGTTCAGTGGAAGGTTGACAACGCTTTGCAATCCGGTAACTCCCAAGA
ATCCGTTACTGAGCAGGATTCTAAGGATTCCACTTACTCATTGTCCTCC
ACTTTGACTTTGTCCAAGGCTGATTACGAGAAGCACAAGGTTTACGCTT
GCGAGGTTACACATCAGGGTTTGTCCTCCCCAGTTACTAAGTCCTTCAA
CAGAGGAGAGTGTTAATAG
SEQ ID NO: 45: α-VEGF light chain (protein)
M R F P S I F T A V L F A A S S A L A D Q I M T Q
S P S S L S A S V G D R V T I T C S A S Q D I S N
Y L N W Y Q Q K P G K A P K V L I Y F T S S L H S
G V P S R F S G S G S G T D F T L T I S S L Q P E
D F A T Y Y C Q Q Y S T V P W T F G Q G T K V E I
K R T V A A P S V F I F P P S D E Q L K S G T A S
V V C L L N N F Y P R E A K V Q W K V D N A L Q S
G N S Q E S V T E Q D S K D S T Y S L S S T L T L
S K A D Y E K H K V Y A C E V T H Q G L S S P V T
K S F N R G E C
SEQ ID NO: 46: α-VEGF heavy chain (DNA)
ATGAGATTCCCATCCATCTTCACTGCTGTTTTGTTCGCTGCTTCTTCTG
CTTTGGCTGAGGTTCAGTTGGTTGAATCTGGTGGTGGATTGGTTCAACC
TGGTGGTTCTTTGAGATTGTCCTGTGCTGCTTCCGGTTACACTTTCACT
AACTACGGAATGAACTGGGTTAGACAGGCTCCAGGTAAAGGATTGGAAT
GGGTTGGATGGATCAACACTTACACTGGTGAACCAACTTACGCTGCTGA
CTTCAAGAGAAGATTCACTTTTTCCTTGGACACTTCCAAGTCCACTGCT
TACTTGCAGATGAACTCCTTGAGAGCTGAGGATACTGCTGTTTACTACT
GTGCTAAGTACCCACACTACTACGGTTCTTCCCACTGGTACTTCGATGT
TTGGGGACAGGGAACTTTGGTTACTGTCTCGAGTGCTTCTACTAAGGGA
CCATCCGTTTTTCCATTGGCTCCATCCTCTAAGTCTACTTCCGGTGGAA
CCGCTGCTTTGGGATGTTTGGTTAAAGACTACTTCCCAGAGCCAGTTAC
TGTTTCTTGGAACTCCGGTGCTTTGACTTCTGGTGTTCACACTTTCCCA
GCTGTTTTGCAATCTTCCGGTTTGTACTCTTTGTCCTCCGTTGTTACTG
TTCCATCCTCTTCCTTGGGTACTCAGACTTACATCTGTAACGTTAACCA
CAAGCCATCCAACACTAAGGTTGACAAGAAGGTTGAGCCAAAGTCCTGT
GACAAGACACATACTTGTCCACCATGTCCAGCTCCAGAATTGTTGGGTG
GTCCATCCGTTTTCTTGTTCCCACCAAAGCCAAAGGACACTTTGATGAT
CTCCAGAACTCCAGAGGTTACATGTGTTGTTGTTGACGTTTCTCACGAG
GACCCAGAGGTTAAGTTCAACTGGTACGTTGACGGTGTTGAAGTTCACA
ACGCTAAGACTAAGCCAAGAGAAGAGCAGTACAACTCCACTTACAGAGT
TGTTTCCGTTTTGACTGTTTTGCACCAGGACTGGTTGAACGGTAAAGAA
TACAAGTGTAAGGTTTCCAACAAGGCTTTGCCAGCTCCAATCGAAAAGA
CTATCTCCAAGGCTAAGGGTCAACCAAGAGAGCCACAGGTTTACACTTT
GCCACCATCCAGAGAAGAGATGACTAAGAACCAGGTTTCCTTGACTTGT
TTGGTTAAAGGATTCTACCCATCCGACATTGCTGTTGAGTGGGAATCTA
ACGGTCAACCAGAGAACAACTACAAGACTACTCCACCAGTTTTGGATTC
TGATGGTTCCTTCTTCTTGTACTCCAAGTTGACTGTTGACAAGTCCAGA
TGGCAACAGGGTAACGTTTTCTCCTGTTCCGTTATGCATGAGGCTTTGC
ACAACCACTACACTCAAAAGTCCTTGTCTTTGTCCCCTGGTTAATGA
SEQ ID NO: 47: α-VEGF heavy chain (protein)
M R F P S I F T A V L F A A S S A L A E V Q L V E
S G G G L V Q P G G S L R L S C A A S G Y T F T N
Y G M N W V R Q A P G K G L E W V G W I N T Y T G
E P T Y A A D F K R R F T F S L D T S K S T A Y L
Q M N S L R A E D T A V Y Y C A K Y P H Y Y G S S
H W Y F D V W G Q G T L V T V S S A S T K G P S V
F P L A P S S K S T S G G T A A L G C L V K D Y F
P E P V T V S W N S G A L T S G V H T F P A V L Q
S S G L Y S L S S V V T V P S S S L G T Q T Y I C
N V N H K P S N T K V D K K V E P K S C D K T H T
C P P C P A P E L L G G P S V F L F P P K P K D T
L M I S R T P E V T C V V V D V S H E D P E V K F
N W Y V D G V E V H N A K T K P R E E Q Y N S T Y
R V V S V L T V L H Q D W L N G K E Y K C K V S N
K A L P A P I E K T I S K A K G Q P R E P Q V Y T
L P P S R E E M T K N Q V S L T C L V K G F Y P S
D I A V E W E S N G Q P E N N Y K T T P P V L D S
D G S F F L Y S K L T V D K S R W Q Q G N V F S C
S V M H E A L H N H Y T Q K S L S L S P G

What is claimed:

1. A method of producing a glycoprotein having reduced O-linked glycosylation comprising:
(a) providing a nucleic acid encoding a glycoprotein;
(b) introducing the nucleic acid into a host cell;
(c) contacting the host cell with an α-1,2-mannosidase enzyme, wherein the α-1,2-mannosidase enzyme comprises an amino acid sequence selected from the group consisting of: SEQ ID NO: 4 (Coccidiodes immitis), SEQ ID NO: 6 (Coccidiodes posadasii), SEQ ID NO: 8 (Penicillium citrinum), SEQ ID NO: 10 (Magnaporthe grisea), SEQ ID NO: 12 (Aspergillus saitoi), SEQ ID NO: 14 (Aspergillus oryzae), and SEQ ID NO: 16 (Chaetomiun globosum); and
(d) isolating the glycoprotein produced by the host cell in the presence of the α-1,2-mannosidase enzyme;
thereby producing a glycoprotein having reduced O-linked glycosylation.

2. The method of claim 1, wherein the host cell is grown for a time sufficient to provide a multiplicity of the host cells having the nucleic acid encoding the glycoprotein before contacting the host cell with the α-1,2-mannosidase enzyme.

3. The method of claim 1, wherein the host cell is grown in the presence of the α-1,2-mannosidase enzyme.

4. The method of claim 1, wherein the nucleic acid encoding the glycoprotein is operably linked to an inducible promoter.

5. The method of claim 4, wherein the host cell is contacted with an inducer of the promoter to induce expression of the glycoprotein for a time before contacting the host cell with the α-1,2-mannosidase enzyme.

6. The method of claim 1, wherein the α-1,2-mannosidase enzyme comprises SEQ ID NO: 4 (Coccidiodes immitis) or a catalytically active fragment thereof.

7. The method of claim 1, wherein the α-1,2-mannosidase enzyme comprises SEQ ID NO: 6 (Coccidiodes posadasii) or a catalytically active fragment thereof.

8. The method of claim 1, wherein the host cell is a fungal cell.

9. The method of claim 1, wherein the host cell is a yeast cell.

10. The method of claim 1, wherein the host cell is selected from the group consisting of K. lactis, Pichia pastoris, Pichia methanolica, and Hansenula polymorpha.

11. The method of claim 1, wherein the host cell is Pichia pastoris.

12. The method of claim 1, wherein the host cell is a yeast or filamentous fungal cell that has been genetically modified to produce glycoproteins with a predominant N-glycan glycoform.

13. The method of claim 1 wherein the host cells have been genetically modified to produce glycoproteins in which the N-glycosylation pattern is human-like or humanized.

14. The method of claim 1, further comprising contacting the host cell with one or more inhibitors of Pmt-mediated O-linked glycosylation.

15. The method of claim 14, wherein the host cell is grown for a time sufficient to provide a multiplicity of the host cells having the nucleic acid encoding the glycoprotein before contacting the host cell with the one or more inhibitors of Pmt-mediated O-linked glycosylation.

16. The method of claim 14, wherein the host cell is grown in the presence of the one or more inhibitors of Pmt-mediated O-linked glycosylation.

17. A method of producing a glycoprotein having reduced O-linked glycosylation comprising:
(a) providing a first nucleic acid encoding a glycoprotein;
(b) providing a second nucleic acid encoding an α-1,2-mannosidase enzyme, wherein the α-1,2-mannosidase enzyme comprises an amino acid sequence selected from the group consisting of: SEQ ID NO: 4 (Coccidiodes immitis), SEQ ID NO: 6 (Coccidiodes posadasii), SEQ ID NO: 8 (Penicillium citrinum), SEQ ID NO: 10 (Magnaporthe grisea), SEQ ID NO: 12 (Aspergillus saitoi), SEQ ID NO: 14 (Aspergillus oryzae), and SEQ ID NO: 16 (Chaetomiun globosum);
(c) introducing the first and second nucleic acid into a host cell;
(d) expressing the first and second nucleic acids in the host cell; and
(e) isolating the glycoprotein produced by the host cell in the presence of the α-1,2-mannosidase enzyme,
thereby producing a glycoprotein having reduced O-linked glycosylation.

18. The method of claim 17, wherein the first nucleic acid encoding the glycoprotein is operably linked to an inducible promoter.

19. The method of claim 17, wherein the second nucleic acid encoding the α-1,2-mannosidase enzyme is operably linked to an inducible promoter.

20. The method of claim 17, wherein the first and the second nucleic acids are operably linked to an inducible promoter.

21. The method of claim 20, wherein expression of the glycoprotein is induced for a time before inducing expression of the α-1,2-mannosidase enzyme.

22. The method of claim 21, wherein expression of the α-1,2-mannosidase enzyme is induced for a time before inducing expression of the glycoprotein.

23. The method of claim 17, wherein the α-1,2-mannosidase enzyme comprises SEQ ID NO: 4 (Coccidiodes immitis) or a catalytically active fragment thereof.

24. The method of claim 17, wherein the α-1,2-mannosidase enzyme comprises SEQ ID NO: 6 (Coccidiodes posadasii) or a catalytically active fragment thereof.

25. The method of claim 17, wherein the host cell is a yeast cell.

26. The method of claim 17, wherein the host cell is selected from the group consisting of K. lactis, Pichia pastoris, Pichia methanolica, and Hansenula polymorpoha.

27. The method of claim 17, wherein the host cell is Pichia pastoris.

28. The method of claim 17, wherein the host cell is a yeast or filamentous fungal cell that has been genetically modified to produce glycoproteins with a predominant N-glycan glycoform.

29. The method of claim 17, wherein the host cells have been genetically modified to produce glycoproteins in which the N-glycosylation pattern is human-like or humanized.

30. The method of claim 17, further comprising contacting the host cell with one or more inhibitors of Pmt-mediated O-linked glycosylation.

31. The method of claim 30, wherein the host cell is grown in culture for a time sufficient to provide a multiplicity of the host cells before contacting the culture with the one or more inhibitors of Pmt-mediated O-linked glycosylation.

32. The method of claim 31, wherein the host cell is grown in the presence of the one or more inhibitors of Pmt-mediated O-linked glycosylation.
